(19) United States
(12) Patent Application Publication
(10) Pub. No.: US 2010/0022999 A1
(43) Pub. Date: Jan. 28, 2010

(54) SYMMETRICAL RF ELECTROSURGICAL SYSTEM AND METHODS

(76) Inventors: David A. Gollnick, San Francisco, CA (US); Greg Leyh, Brisbane, CA (US)

Correspondence Address: Cutera c/o MoFo, 425 Market Street, San Francisco, CA, 94105-2482, US

(21) Appl. No.: 12/330,032

(22) Filed: Dec. 8, 2008

Related U.S. Application Data
(60) Provisional application No. 61/083,483, filed on Jul. 24, 2008.

(51) Int. Cl. A61B 18/18; A61B 18/14

(52) U.S. Cl. 606/33; 606/41

(57) ABSTRACT

Systems, apparatus, and methods for treating a patient's tissue via electric energy delivered concurrently from a first electrode of a first handpiece and a second electrode of a second handpiece. Each of the first and second handpieces may have the same or similar structure and may be separately manipulable to different locations on the patient's skin to allow the rapid treatment of target tissue(s) at various regions of the patient's body. The first and second handpieces may each be coupled to an electrosurgical generator configured for providing first and second AC voltages of equal magnitude and opposite polarity to the first and second electrodes, respectively. The first and second electrodes may each comprise a spiral inductor.

### CROSS-REFERENCE TO RELATED APPLICATIONS

This application claims the benefit of U.S. Provisional Application No. 61/083,483, filed Jul. 24, 2008, (Attorney Docket No. ALTU 3500), the disclosure of which is incorporated by reference herein in its entirety.

### FIELD OF THE INVENTION

The present invention generally relates to RF electrosurgical systems and methods for treating a patient's tissues.

### BACKGROUND OF THE INVENTION

Various forms of electrosurgery are now widely used for a vast range of surgical procedures. Conventionally, electrosurgery has been considered to be within one of two major categories, namely monopolar and bipolar, according to the electrode configuration of the electrosurgical system which determines the path of electrical energy flow vis-à-vis the patient and the surgical site. In the bipolar configuration, both the active electrode and the return electrode are located adjacent to a target tissue of the patient, i.e., the electrodes are in close proximity to each other, and current flows between the electrodes locally at the surgical site.

In monopolar electrosurgery, the active electrode is again located at the surgical site; however, the return electrode, which is typically much larger than the active electrode, is placed in contact with the patient at a location on the patient's body that is remote from the surgical site. Current from an electrosurgical generator typically flows through an active electrode and into target tissue of the patient. The current then passes through the patient's body to the return electrode where it is collected and returned to the generator. In monopolar electrosurgery, the return electrode is typically accommodated on a device which may be referred to as a dispersive pad, and the return electrode may also be known as the dispersive-, patient-, neutral-, or grounding electrode.

A disadvantage of monopolar electrosurgery is the risk of burns on the patient's body at the location of the return pad as well as at various other sites on the patient's body which may provide an alternative path to ground. In the case of a solid return pad, inadequate surface area of the return pad, or excessive electric current density at the edges of the return pad, may cause a return pad patient burn. At the same time, in an unbalanced electrosurgical system, e.g., using an active electrode and a dispersive ground pad as return electrode, current leakage to ground via structures or equipment surrounding the patient may present a risk of an alternate site patient burn. Thus, any excessive concentration of current density at the return pad or alternate site may inadvertently cause a severe burn to a non-targeted tissue of the patient's body.

The proportion of children, adolescents, and adults who are overweight or obese is increasing. The number of overweight people has doubled in the last two to three decades, and such increases are found in all age, race, and gender groups. Excess body fat may accumulate in overweight and obese individuals on various parts of the body, including the abdomen, thighs, buttocks, face, neck, legs, and arms.

Cellulite is a common skin condition related to the accumulation of excess subcutaneous fat (adipose tissue) within fibrous septae. Irregularities in the structure of the fibrous septae can create the appearance of cellulite, which is typically seen as an unsightly irregular, dimpled skin surface. Cellulite is often found in abundance in overweight and obese individuals, e.g., on the thighs, hips, and buttocks.

There is a demand for apparatus and procedures that will reduce the overall volume of adipose tissue and/or reshape subcutaneous fat. There is also a demand for treatments that will decrease the appearance of cellulite for cosmetic purposes.

Prior art interventions for decreasing or reshaping adipose tissue include liposuction and lipoplasty, massage, low level laser therapy, and external topical compositions, such as “cosmeceuticals,” or a combination of such treatments. Liposuction and lipoplasty are invasive surgical techniques in which subcutaneous fat is excised and/or suctioned from the body. These procedures may be supplemented by the application to the targeted adipose tissue of various forms of energy to emulsify the fat prior to its removal, e.g., by suction.

Although liposuction and lipoplasty can effectively remove subcutaneous fat, the invasive nature of these procedures presents the inherent disadvantages of surgery, including high cost and extended recovery times, as well as the associated risks such as infection, excessive bleeding, and trauma.

Non-invasive interventions for subcutaneous fat reduction, or diminution of the appearance of cellulite, including massage and low-level laser therapy, are significantly less effective than surgical intervention.

Some cosmetic skin treatments effect dermal heating by applying radiofrequency (RF) energy to the skin using surface electrodes. The local heating is intended to tighten the skin by producing thermal injury that changes the ultrastructure of collagen in the dermis, and/or results in a biological response that changes the dermal mechanical properties. The literature has reported some atrophy of sub-dermal fat layers as a complication to skin tightening procedures.

During electrosurgical procedures that target subcutaneous fat, the depth of muscle tissue below the surface of the skin may greatly influence the distribution of electric currents, and therefore the heating distribution within the tissues. Prior art apparatus and methods have not adequately addressed electrode configuration in relation to electric current distribution in subcutaneous tissue, e.g., as influenced by variations in the thickness or depth of skeletal muscle underlying targeted subcutaneous fat.

U.S. Pat. No. 6,488,678 to Sherman discloses apparatus including a catheter having an array of electrodes at the catheter distal end, and adapted to position the electrodes at a biological site. A backplate is positioned proximal to the biological site, such that the biological site is interposed between the proximal backplate and the distal electrode array. Power provided to the distal electrodes has a duty cycle with on and off periods. During a first segment of the on period, energy flows between the backplate and a distal electrode, while during a second segment of the on period, energy flows between the electrodes of the array. The flow of energy can be controlled by adjusting the phase angle of the power.

U.S. Pat. No. 6,635,056 to Kadhiresan et al. discloses a system including a catheter for use in ablation therapy of cardiac tissue, in which the system uses controllable differences in amplitude of power signals to establish repetitive bipolar current flow between sets of electrodes, and a backplate to establish unipolar current flow.

U.S. Pat. No. 7,151,964 to Desai discloses a multi-electrode catheter for ablation of endocardiac tissues. The electrodes are adapted for being collapsed for introducing the catheter into the patient's body, and for being fanned out into an array during ablation of tissue, such as endomyocardium. In a preferred embodiment of the '964 patent, a two-phase RF power source is used with an orthogonal electrode catheter array comprising a central electrode and four peripheral electrodes. The central electrode is connected to ground voltage of the power supply; and the peripheral electrodes form two diagonal pairs connected to two individually phased voltages.

US Patent Application Publication No. 20060036300 (Kreindel) discloses lipolysis apparatus having one or more protruding terminal electrodes. In methods of Kreindel, a region of tissue may be deformed, and the electrodes may contact both deformed and non-deformed skin.

U.S. Patent Application Publication No. 20070203482 (Ein Gal) discloses a system including at least two target electrodes, at least one return electrode, and at least two RF power sources in electrical communication with the electrodes. Each target electrode defines a separate monopolar energy delivery channel, the at least one return electrode being common to both channels. The target electrodes are operable in a bipolar mode. A waveform manipulator controls and manipulates RF energy waveforms to the target electrodes to selectively provide pure monopolar, pure bipolar and a blend of monopolar and bipolar modes of energy delivery for tissue ablation.

It can be seen that there is a need for an electrosurgical system that decreases the risk of alternate site patient burns and at the same time eliminates the risk of return pad patient burns. There is a further need for an effective modality by which subcutaneous fat tissue may be non-invasively reshaped, and/or removed for improving the appearance of human skin or for sculpting the human body.

### SUMMARY OF THE INVENTION

According to one aspect of the invention, a system for treating a patient comprises an electrosurgical generator, a first handpiece coupled to the electrosurgical generator, and a second handpiece coupled to the electrosurgical generator. The system is configured for providing a first AC voltage to the first handpiece and for providing a second AC voltage to the second handpiece. The first handpiece and the second handpiece are manipulable separately from each other.

According to another aspect of the invention there is provided a balanced electrosurgical system for treating a patient, wherein the system comprises an electrosurgical generator, a first handpiece having a first electrode electrically coupled to the electrosurgical generator, and a second handpiece having a second electrode electrically coupled to the electrosurgical generator. The electrosurgical generator is configured for providing a first AC voltage to the first electrode, the electrosurgical generator is further configured for concurrently providing a second AC voltage to the second electrode, and the first and second AC voltages are of equal magnitude and opposite polarity.

According to still another aspect of the invention, an electrosurgical system for treating a patient comprises an electrosurgical generator, a first electrode coupled to the electrosurgical generator via a first cable, and a second electrode coupled to the electrosurgical generator via a second cable. The first and second cables are manipulable independently of each other, and the electrosurgical generator is configured for providing a first AC voltage to the first electrode. The electrosurgical generator is further configured for concurrently providing a second AC voltage to the second electrode. The first and second AC voltages have a phase difference of about 180°. The system is configured for controlling delivery of electrical power from each of the first and second electrode to a target tissue of the patient. The first and second electrode are separately movable with respect to each other, and each of the first and second electrodes comprises a spiral inductor.

According to yet another aspect of the invention, a system for monitoring treatment of a patient comprises at least one handpiece including an electrode disposed within a void of the handpiece, a vacuum unit in fluid communication with the handpiece, and at least one pressure sensor disposed within the void. The handpiece is configured for applying suction to the skin of the patient via the vacuum unit. The pressure sensor is configured for sensing pressure values within the void, and the system is configured for monitoring patient contact with the electrode via the sensed pressure values.

According to a further aspect of the invention, a handpiece for treating a patient comprises a shell, and a planar electrode disposed at a substantially central location within the shell, wherein the electrode comprises a spiral inductor.

According to still another aspect of the invention, a handpiece for treating a patient comprises a shell including a central planar portion and a planar electrode recessed within the shell, wherein the electrode is disposed at a substantially central location within the shell, and the electrode is disposed substantially parallel to the central planar portion. The shell includes at least one suction port and a collar portion extending distally from the central planar portion. The shell is frusto-pyramidal or frusto-conical and defines a void within the handpiece. The handpiece is configured for applying suction, via the suction port, to tissue of the patient. The handpiece is further configured for receiving the tissue of the patient within the void, such that an external surface of the skin contacts the electrode.

According to still a further aspect of the invention, there is provided a method for treating a patient, wherein the method comprises providing a first AC voltage to a first electrode of an electrosurgical system, and concurrently providing a second AC voltage to a second electrode of the electrosurgical system. The first and second AC voltages are of substantially equal magnitude and opposite polarity, whereby a potential difference is provided between the first and second electrodes. The method further comprises applying electrical energy to a target tissue of the patient via the first and second electrodes. The electrical energy is sufficient to remove or modify at least a portion of the target tissue.

According to yet another aspect of the invention, a method for treating a patient comprises providing an electrosurgical system having a first handpiece and a second handpiece. The first handpiece has a first electrode and the second handpiece has a second electrode. Each of the first handpiece and the second handpiece is configured for contacting the skin of the patient. The method further comprises disposing the first handpiece at a first skin location on the patient, such that the external surface of the first electrode contacts the external surface of the skin at the first skin location; and disposing the second handpiece at a second skin location on the patient, such that the external surface of the second electrode contacts the external surface of the skin at the second skin location. The method still further comprises providing a first AC voltage to the first electrode, and concurrently providing a second AC voltage to the second electrode, wherein the first and second AC voltages have a phase difference of about 180°, whereby a potential difference is provided between the first and second electrodes. The method still further comprises applying electrical energy to a target tissue of the patient via the first and second electrodes, wherein the electrical energy is sufficient to remove or modify at least a portion of the target tissue.

According to yet a further aspect of the invention, there is provided a method for making a handpiece, the method comprising providing a shell for the handpiece, providing an electrode for the handpiece, and disposing the electrode at a substantially central location within the shell. The electrode comprises a spiral inductor, and the electrode is at least substantially planar.

According to yet a further aspect of the invention, a method for making a multi-layered spiral inductor comprises forming a first spiral and a second spiral, aligning the first spiral with the second spiral, and electrically interconnecting the first and second spirals. Each of the first and second spirals is at least substantially planar, and each of the first and second spirals comprises a spiral trace of electrically conductive metal.

According to still a further aspect of the invention, there is provided a method for monitoring patient-electrode contact during an electrosurgical procedure, the method comprising contacting a patient's body with a handpiece, wherein the handpiece includes a shell defining a void, a substantially planar electrode disposed in the void, and at least one pressure sensor configured for sensing pressure values within the void, and wherein the shell includes at least one suction port in communication with the void. The method further comprises applying a vacuum to the suction port, wherein an area of skin of the patient's body is drawn into the void such that the skin contacts the electrode; sensing pressure values within the void via the pressure sensor; and monitoring contact between the electrode and the skin via the sensed pressure values.

According to still a further aspect of the invention, there is provided a method for controlling skin temperature during an electrosurgical procedure, comprising contacting a patient's body with a handpiece, wherein the handpiece includes a shell defining a void, an electrode disposed in the void, at least one suction port in communication with the void, at least one temperature sensor configured for sensing temperature values of the skin, wherein the temperature sensor is disposed adjacent to the electrode, and a cooling unit configured for cooling the skin. The method further comprises applying a vacuum to the suction port, wherein an area of the skin of the patient's body is drawn into the void such that the skin contacts both the electrode and the temperature sensor; sensing temperature values of the skin via the temperature sensor; and adjusting a voltage applied to the cooling unit in response to the sensed temperature values.

These and other features, aspects, and advantages of the present invention may be further understood with reference to the drawings, description, and claims which follow.

### BRIEF DESCRIPTION OF THE DRAWINGS

FIGS. 1A-B each schematically represents an electrosurgical system for treating a patient, according to the instant invention;

FIG. 2A schematically represents an electrosurgical system for treating a patient via first and second AC voltages provided to first and second electrodes, according to another embodiment of the invention;

FIG. 2B schematically represents the first and second AC voltages of the embodiment of FIG. 2A;

FIG. 3A is a block diagram schematically representing an electrosurgical system for treating a patient via first and second AC voltages, according to another embodiment of the invention;

FIG. 3B is a block diagram schematically representing an electrosurgical system for treating a patient via first and second AC voltages, according to another embodiment of the invention;

FIG. 4A is a block diagram schematically representing an electrosurgical system for treating a patient via first and second handpieces, according to another aspect of the invention;

FIG. 4B is a block diagram schematically representing an electrosurgical system including a control unit in communication with a handpiece having at least one temperature sensor, according to another aspect of the invention;

FIG. 4C is a block diagram schematically representing an electrosurgical system for treating a patient via first and second handpieces including a pressure sensor, according to another aspect of the invention;

FIG. 5 is a block diagram schematically representing a handpiece for an electrosurgical system, according to another embodiment of the invention;

FIG. 6A is a schematic representation of a handpiece, in plan view as seen from above, according to another aspect of the invention;

FIGS. 6B-C each show a sectional view of the handpiece of FIG. 6A, as seen along the line 6B/C-6B/C of FIG. 6A, according to two different embodiments of the invention;

FIG. 6D is a plan view of the handpiece of FIG. 6C, as seen along the line 6D-6D of FIG. 6C;

FIG. 7A is a sectional side view of a handpiece including at least one pressure sensor, according to another embodiment of the invention;

FIG. 7B schematically represents the handpiece of FIG. 7A, as seen along the line 7B-7B of FIG. 7A;

FIG. 8A schematically represents an electrosurgical system including two electrode-bearing pads, according to another embodiment of the invention;

FIG. 8B schematically represents a conformable electrode-bearing pad, as seen in side view in relation to a portion of a patient's body, according to another embodiment of the invention;

FIG. 9 schematically represents a spiral of electrically conductive material for forming an electrode, as seen in plan view, according to another embodiment of the invention;

FIG. 10A schematically represents a spiral inductor for an electrode, as seen in plan view, according to an embodiment of the invention;

FIG. 10B schematically represents a spiral inductor for an electrode, as seen in plan view, according to another embodiment of the invention;

FIG. 11 is a sectional view of a portion of the spiral inductor of FIGS. 10A-B, as seen along the line 11-11 of FIGS. 10A-B, according to an embodiment of the invention;

FIG. 12A is a schematic sectional view of a spiral inductor having a plurality of spirals, showing electrical connections between each spiral, according to one aspect of the invention;

FIG. 12B is a schematic sectional view of a spiral inductor having a plurality of spirals, showing electrical connections between each spiral, according to the invention;

FIG. 13 is a schematic sectional view of a handpiece including an electrode comprising a spiral inductor, according to another embodiment of the invention;

FIG. 14A is a flow chart schematically representing steps in a method for treating a patient, according to another embodiment of the invention;

FIG. 14B is a flow chart schematically representing steps in a method for treating a patient, according to another embodiment of the invention;

FIG. 15 is a flow chart schematically representing steps in a method for making a handpiece for an electrosurgical system, according to another embodiment of the invention;

FIG. 16A is a flow chart schematically representing steps in a method for making a spiral inductor, according to another embodiment of the invention;

FIG. 16B is a flow chart schematically representing steps in a method for making a multi-layer spiral inductor, according to another embodiment of the invention;

FIG. 16C is a flow chart schematically representing steps in a method for making a multi-layer spiral inductor, according to another embodiment of the invention;

FIG. 17A schematically represents a handpiece, as seen from the side, showing a void of the handpiece in relation to a target region of skin of a patient, according to one aspect of the invention;

FIG. 17B schematically represents the handpiece of FIG. 17A showing a target tissue of the patient disposed within a void of the handpiece, according to the invention;

FIG. 18 is a flow chart schematically representing steps in a method for monitoring patient-electrode contact during an electrosurgical procedure, according to another embodiment of the invention; and

FIG. 19 is a flow chart schematically representing steps in a method for controlling skin temperature during an electrosurgical procedure, according to another embodiment of the invention.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

Broadly, the present invention provides methods and apparatus for treating or selectively heating a target tissue of a patient in a non-invasive procedure. As a non-limiting example, the instant invention may be used to selectively heat, remove, and or sculpt adipose tissue, such as may be present in subcutaneous fat and/or cellulite.

Apparatus of the present invention may include a first handpiece having a first electrode and a second handpiece having a second electrode. The first and second handpieces may each be coupled to an electrosurgical generator configured for providing first and second AC voltages to the first and second electrodes, respectively. The generator may be configured for providing a phase difference of approximately 180° between the first and second electrodes, and the first and second AC voltages may be of substantially equal magnitude and opposite polarity. Each of the first and second electrodes may be used to selectively heat (i.e., treat) tissue, wherein each of the first and second electrodes may function as an active electrode to simultaneously treat tissue at two separate locations on the patient's body.

The present invention provides a balanced electrosurgical system that decreases the risk of alternate site patient burns due to current leakage to ground. The present invention also eliminates the use of a dispersive return electrode (ground pad). Unlike devices of the prior art, the present invention further provides an electrosurgical system wherein two separate handpieces can be controlled, via a single electrosurgical generator, with respect to parameters of electrical energy delivered by the first and second electrodes to the patient's tissue. The present invention also provides an electrosurgical system wherein both of the first and second handpieces can be actively cooled by the incorporation of a cooling unit, such as a thermoelectric cooler, in each handpiece. The present invention also provides an electrosurgical system wherein both of the first and second handpieces can be separately attached to the patient's skin by the application of suction to each handpiece. This is in contrast to conventional devices and methods of the prior art that use a passive dispersive pad as a return electrode, wherein the dispersive return pad must dissipate electric current density over a relatively large area, the return pad is not controlled with respect to power delivery, the return pad is not actively cooled, and the return pad relies on a layer of adhesive for contact of the return pad on the skin. Apparatus of the present invention may be further distinguished from prior art devices by having a pair of handpieces, each handpiece including an electrode comprising a spiral inductor.

In further contrast to prior art devices and methods, each of the first and second handpieces of the inventive electrosurgical apparatus may be separately manipulable or movable to different locations on the patient's body to provide various separation distances between the first and second handpieces, thereby allowing the treatment of target tissues at different depths and different locations on the patient's body. In still further contrast to prior art devices and methods, each of the first and second handpieces may have the same or similar structure, components, and/or configuration.

### Balanced Electrosurgical Systems

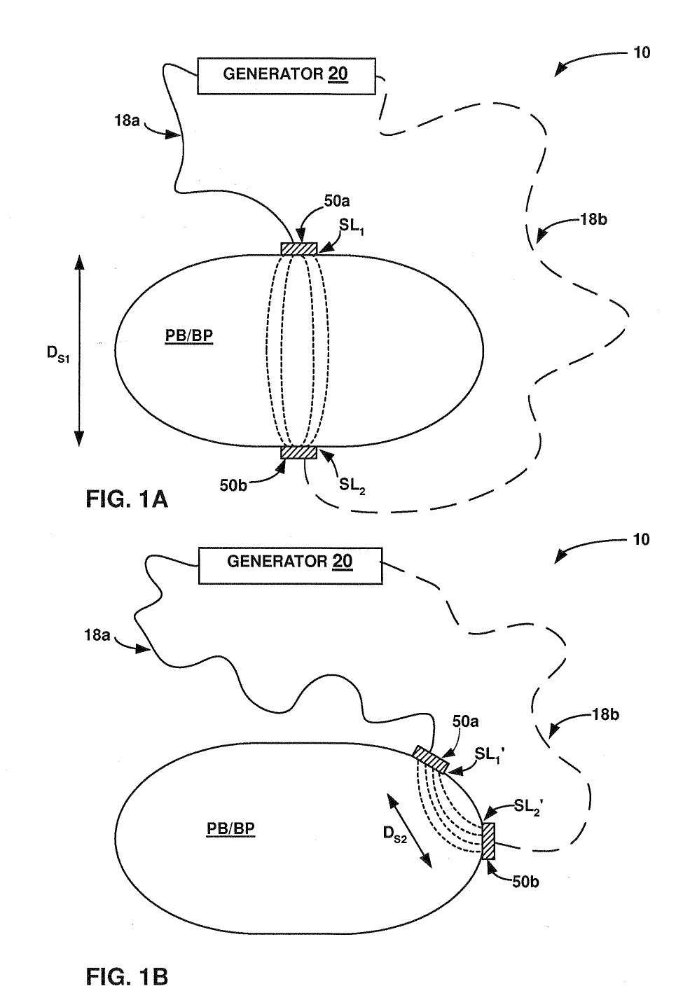

FIGS. 1A-B each schematically represents an electrosurgical system for treating a patient, according to the instant invention. System 10 may include an electrosurgical generator 20, a first handpiece 50a, and a second handpiece 50b. First handpiece 50a and second handpiece 50b are shown in relation to a section through a patient's body or body part, PB/BP. System 10 may be configured for flow of electric current through the patient's tissue disposed between first handpiece 50a and second handpiece 50b. Electric current distribution in FIGS. 1A-B may be represented by broken lines extending between first handpiece 50a and second handpiece 50b. As non-limiting examples, the patient's body or body part of FIGS. 1A-B may represent the patient's torso, neck, an arm, a leg, or the buttock(s), and the like.

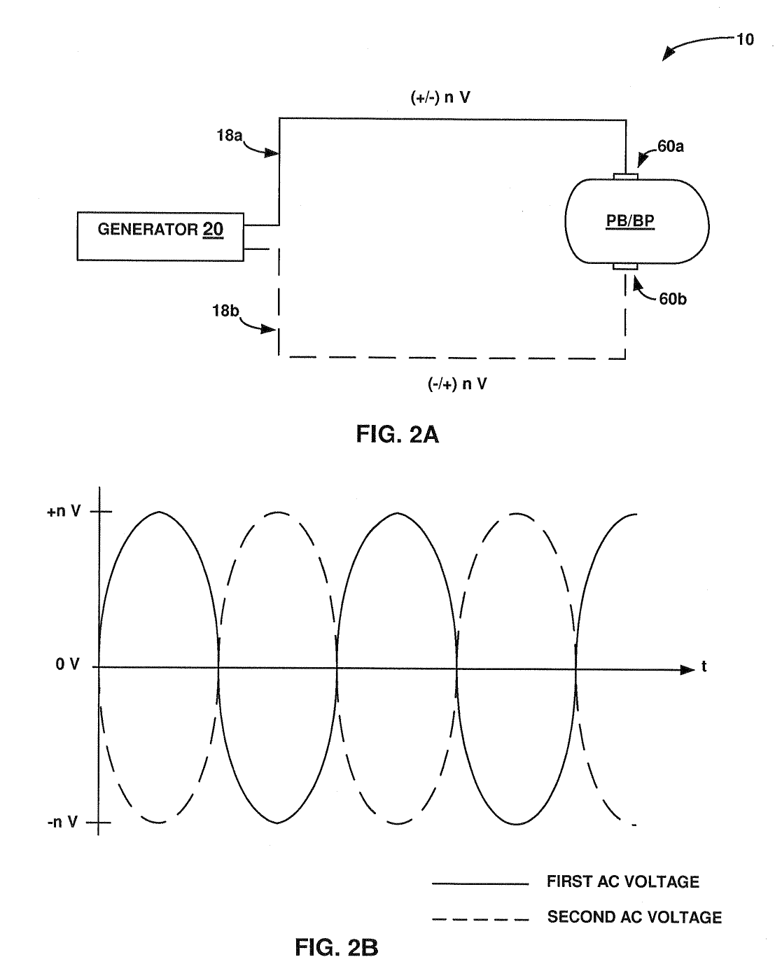

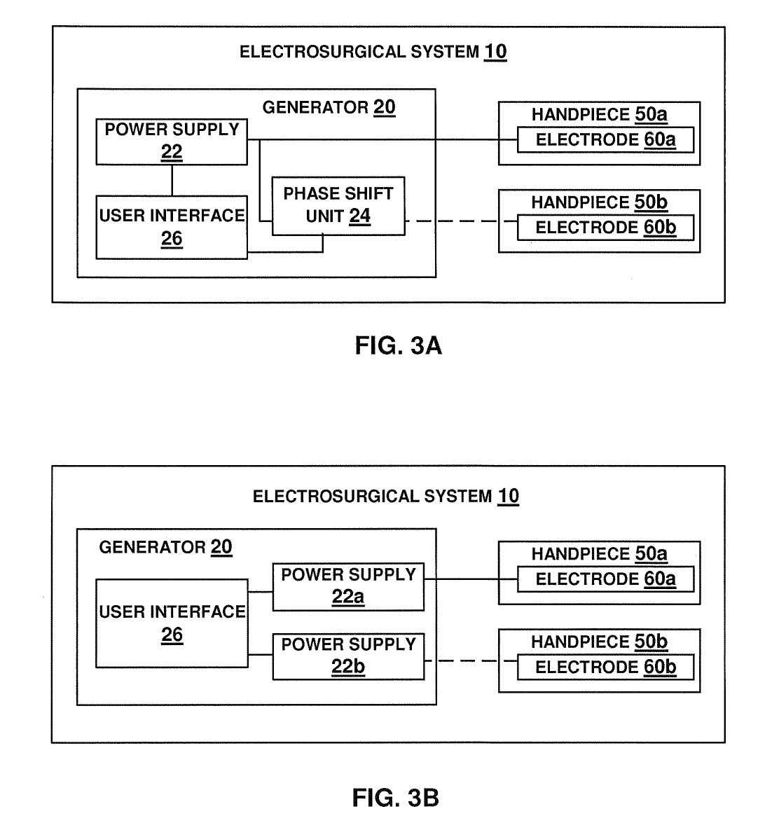

First handpiece 50a may be electrically coupled to electrosurgical generator 20 via a first cable 18a, and second handpiece 50b may be separately electrically coupled to electrosurgical generator 20 via a second cable 18b, wherein first and second cables 18a, 18b may be movable independently of each other. First handpiece 50a may include a first electrode 60a, and second handpiece 50b may include a second electrode 60b (see, e.g., FIGS. 3A-B, and 4B). Electrosurgical generator 20 may be configured for concurrently providing a first AC voltage to first handpiece 50a and a second AC voltage to second handpiece, wherein the first and second AC voltages may be about 180° out of phase (see, e.g., FIG. 2B). According to one aspect of the invention, each of first electrode 60a and second electrode 60b may function as an active electrode to treat a different area of the patient's body at the same time; that is to say, first electrode 60a may be used to treat a first area of the patient's body and, at the same time, second electrode 60b may be used to treat a second area of the patient's body.

Each of first and second handpieces 50a, 50b may be configured for contacting an area of an intact external surface of skin of the patient's body or body part. In an embodiment, such an area of skin may typically be at least about 10 cm², usually at least about 20 cm², and often at least about 50 cm². In an embodiment, each of first and second handpieces 50a, 50b may be configured for being affixed or adhered to an external skin surface of a patient. First and second handpieces 50a, 50b may have similar or substantially identical structure, i.e., first and second handpieces 50a, 50b may have substantially the same or similar components in substantially the same or similar configuration. In an embodiment, first and second handpieces 50a, 50b may be of substantially the same or similar size and shape. In another embodiment, first and second handpieces 50a, 50b may be of at least substantially similar structure, but of different size and/or shape. As an example, the size and/or shape of first and second handpieces 50a, 50b may be varied according to various region(s) of the patient's body to be treated.

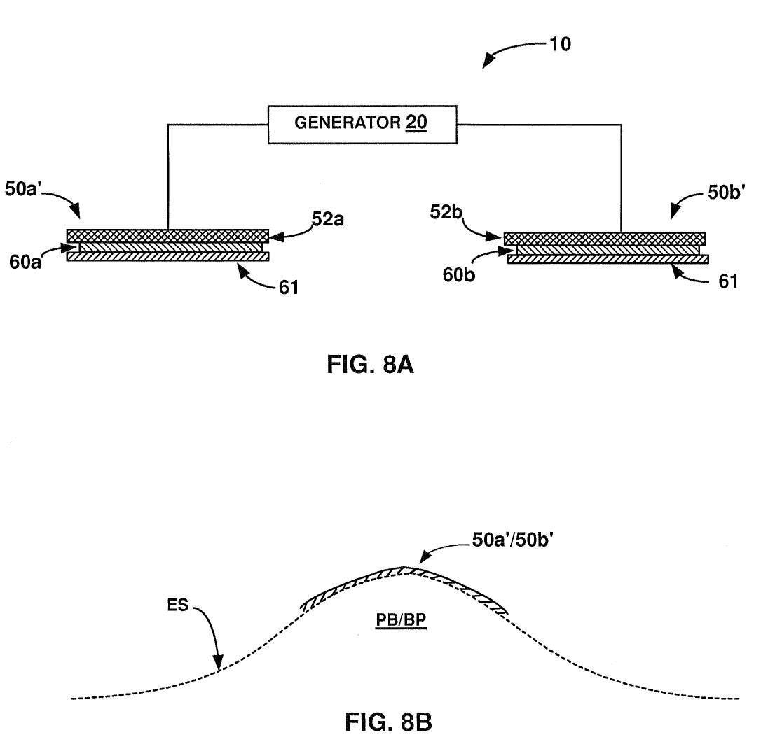

Each of first and second handpieces 50a, 50b may be separately or independently movable with respect to each other. For example, first and second handpieces 50a, 50b may be manipulable separately from each other, and each may be disposed at various selected locations on the patient's body. In another embodiment, the invention may comprise an electrosurgical system 10 having first and second electrode-bearing pads 50a′, 50b′, respectively, in lieu of first and second handpieces 50a, 50b (see, e.g., FIG. 8A).

With reference to FIG. 1A, first handpiece 50a may be disposed at a first location, SL₁, on the skin of the patient, and second handpiece 50b may be disposed at a second location, SL₂, on the skin of the patient, wherein first and second handpieces 50a, 50b may be separated by a first separation distance, Dₛ₁. Herein, locations on the skin of the patient may be referred to as “skin locations,” viz. first skin location (SL₁) and second skin location (SL₂). When first and second AC voltages are provided to first and second handpieces 50a, 50b, current flows therebetween to treat tissue disposed between first and second handpieces 50a, 50b.

FIG. 1A depicts first and second handpieces 50a, 50b as being diametrically opposite with respect to the patient's body or body part, to provide a relatively large separation distance therebetween. In contrast, FIG. 1B shows first handpiece 50a disposed at first skin location, SL₁′, and second handpiece 50b disposed at second skin location, SL₂′, such that first and second handpieces 50a, 50b may be separated by a second separation distance, Dₛ₂, wherein Dₛ₂>Dₛ₂. Thus, the separation distance between first and second skin locations of first and second handpieces 50a, 50b, respectively, can be varied from treatment to treatment or during treatment of a single region of the patient. A relatively large separation distance (Dₛ₁) between first and second handpieces 50a, 50b (e.g., FIG. 1A) may result in relatively deep electric current distribution within the tissue. In general, increased separation distance between first and second handpieces 50a, 50b, may result in increased depth of treatment, e.g., relative to the skin surface.

FIG. 2A schematically represents an electrosurgical system for treating a patient via first and second AC voltages provided to first and second electrodes, according to another embodiment of the invention. System 10 may include an electrosurgical generator 20, a first electrode 60a, and a second electrode 60b. First and second electrodes 60a, 60b may be electrically coupled to generator 20 via first and second cables, 18a, 18b, respectively. First and second cables 18a, 18b may be independently movable with respect to each other. Generator 20 may be configured for providing a first AC voltage to first electrode 60a, and generator 20 may be further configured for concurrently providing a second AC voltage to second electrode 60b. Electrosurgical generator may be configured for providing a phase difference between the first and second AC voltages. In an embodiment, the phase difference between the first and second AC voltages may be about 180°.

FIG. 2B schematically represents the first and second AC voltages of the embodiment of FIG. 2A. Each of the first and second AC voltages may be in the form of a sine wave, and the first and second AC voltages may be 180° out of phase. The first and second AC voltages may be equal, or at least substantially equal, in magnitude and of opposite polarity. Each of the first and second AC voltages may have a peak-to-peak voltage of 2 n Volts (V). In an embodiment, the peak-to-peak voltage of each of first and second AC voltages may typically be in the region of from about 100 to 1200 V, usually from about 200 to 1000 V, and often from about 400 to 800 V.

Each of first electrode 60a and second electrode 60b may be at least substantially planar. System 10 (see, e.g., FIG. 2A) may be configured for independent manipulation of first electrode 60a and second electrode 60b, such that each of first and second electrodes 60a, 60b may be disposed at various locations on the skin of a patient. In this way, tissue at various regions of the patient's body can be targeted, and furthermore, tissue at different depths beneath the skin of the patient may be targeted by varying the separation distance between the first and second electrodes 60a, 60b (see, e.g., FIGS. 1A-B).

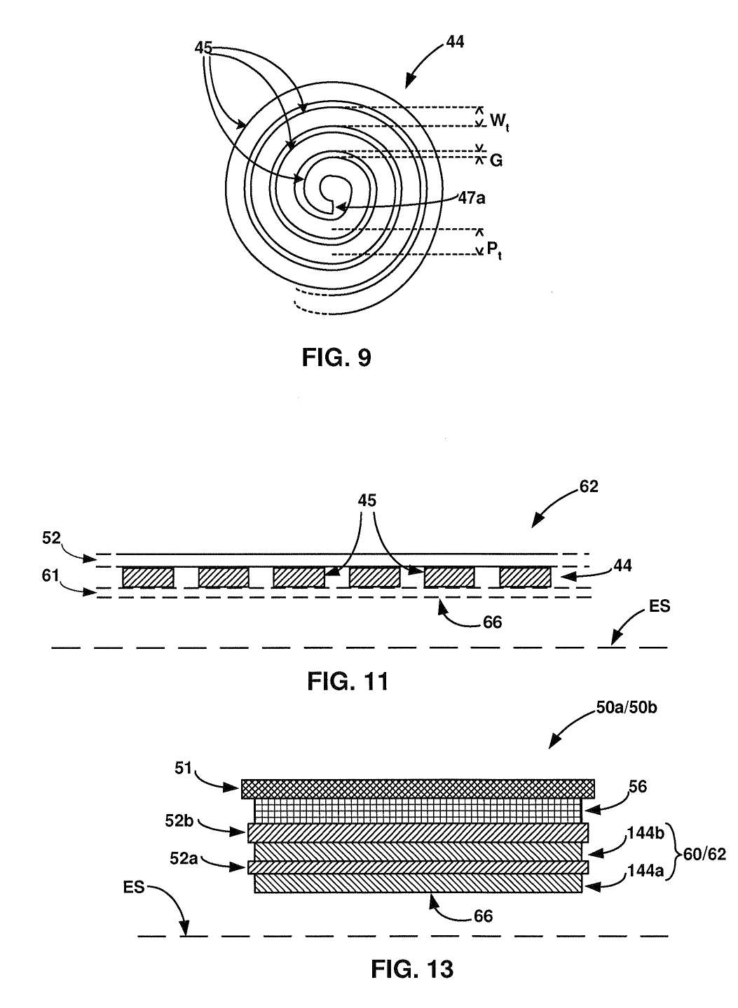

Each of first electrode 60a and second electrode 60b may be configured for contacting an external surface area of skin of the patient. In an embodiment, such an area of skin contacted by each of first electrode 60a and second electrodes 60b, at a single skin location, may typically be at least about 10 cm², usually at least about 20 cm², and often at least about 50 cm². In an embodiment, each of first electrode 60a and second electrode 60b may comprise a spiral inductor, wherein each spiral inductor may comprise at least one spiral of electrically conductive metal (see, e.g., FIGS. 9-12B).

FIG. 3A is a block diagram schematically representing an electrosurgical system for treating a patient via first and second AC voltages, according to another embodiment of the invention. System 10 of FIG. 3A may include an electrosurgical generator 20, a first handpiece 50a, and a second handpiece 50b. First handpiece 50a may include a first electrode 60a, and second handpiece 50b may include a second electrode 60b. First and second electrodes 60a, 60b may be affixed to first and second handpieces 50a, 50b, respectively.

Generator 20 may include a power supply 22, a phase shift unit 24, and a user interface 26. User interface 26 may be electrically coupled to or in signal communication with power supply 22 and phase shift unit 24. Power supply 22 may be configured for providing a first AC voltage. System 10 may be configured for providing the first AC voltage to first electrode 60a. The first AC voltage may also be provided (input) to phase shift unit 24. Phase shift unit 24 may be configured for receiving the first AC voltage and for shifting the phase of the first AC voltage to provide a second AC voltage, such that a significant phase difference exists between the first and second AC voltages. System 10 may be configured for selecting, e.g., via user interface 26, an extent or degree of phase difference. In an embodiment, the second AC voltage may be shifted about 180° out of phase with respect to the first AC voltage.

Each of the first and second AC voltages may be of high frequency, e.g., in the radiofrequency (RF) range. In an embodiment, the frequency of the first and second AC voltages may be in the range of from about 0.1 MHz to 6 MHz, usually from about 0.2 MHz to 5 MHz, and often from about 0.5 MHz to 4 MHz. The first and second AC voltages may have the same frequency. The actual frequency, as well as other parameters, of the first and second AC voltages may be selected by a user of system 10, e.g., via user interface 26. For example, user interface 26 may be used to select an output signal requirement for each of first and second AC voltages, e.g., with respect to one or more parameters such as power, voltage, phase difference, and frequency. In an embodiment, the first and second AC voltages may be of opposite polarity and equal magnitude.

FIG. 3B is a block diagram schematically representing an electrosurgical system for treating a patient via first and second AC voltages, according to another embodiment of the invention. System 10 of FIG. 3B may include an electrosurgical generator 20, a first handpiece 50a and a second handpiece 50b. First handpiece 50a may include a first electrode 60a and second handpiece 50b may include a second electrode 60b.

Generator 20 of FIG. 3B may include a first power supply 22a, a second power supply 22b, and a user interface 26. User interface 26 may be electrically coupled to or in signal communication with both first power supply 22a and second power supply 22b. First power supply 22a may be configured for providing a first AC voltage to first electrode 60a. Second power supply 22b may be configured for providing a second AC voltage to second electrode 60b, wherein a significant phase difference may exist between the first and second AC voltages. System 10 may be configured for selecting a degree of phase difference via user interface 26. In an embodiment, the second AC voltage may be shifted about 180° out of phase with respect to the first AC voltage. Each of the first and second AC voltages may be of high frequency, e.g., in the radiofrequency (RF) range, substantially as described with reference to FIG. 3A. In an embodiment, the frequency of the first and second AC voltages may be in the range of from about 0.1 MHz to 6 MHz, usually from about 0.2 MHz to 5 MHz, and often from about 0.5 MHz to 4 MHz. The first and second AC voltages may have the same frequency. The actual frequency, as well as other parameters, of the first and second AC voltages may be selected by a user of system 10, e.g., via user interface 26, substantially as described with reference to FIG. 3A. In an embodiment, the first and second AC voltages may be of equal magnitude and opposite polarity.

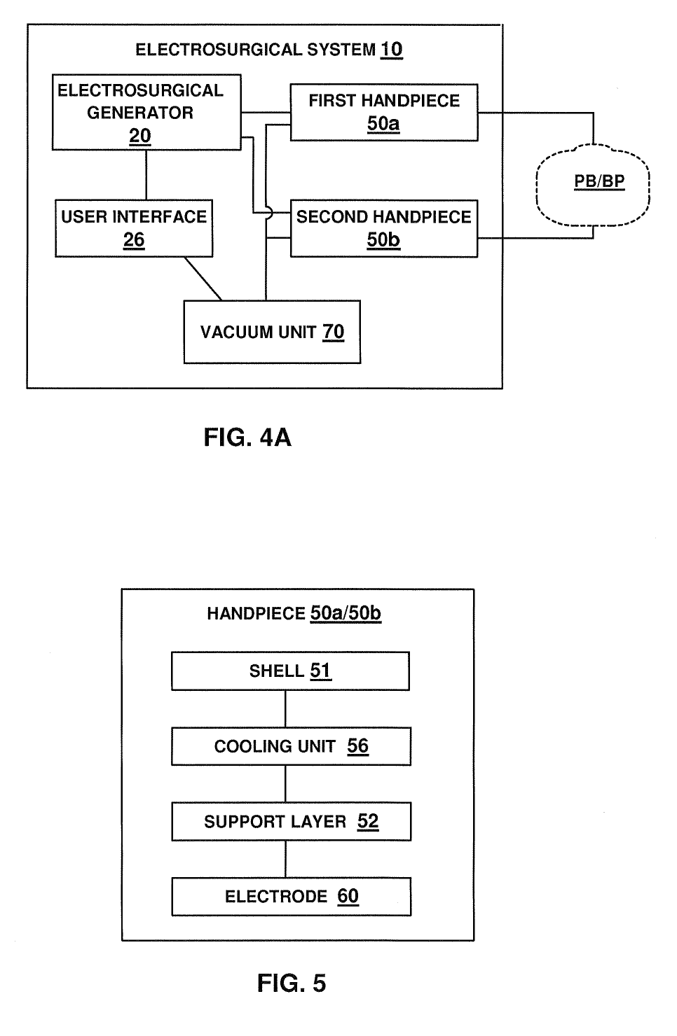

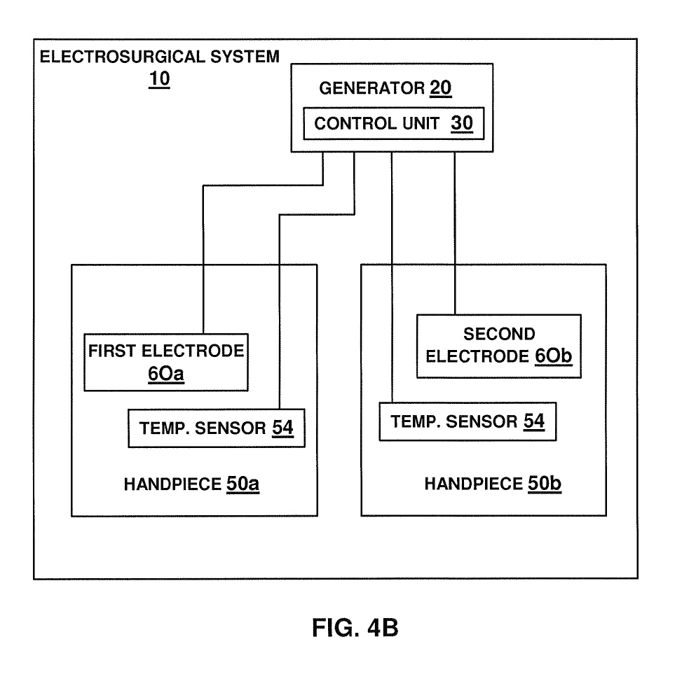

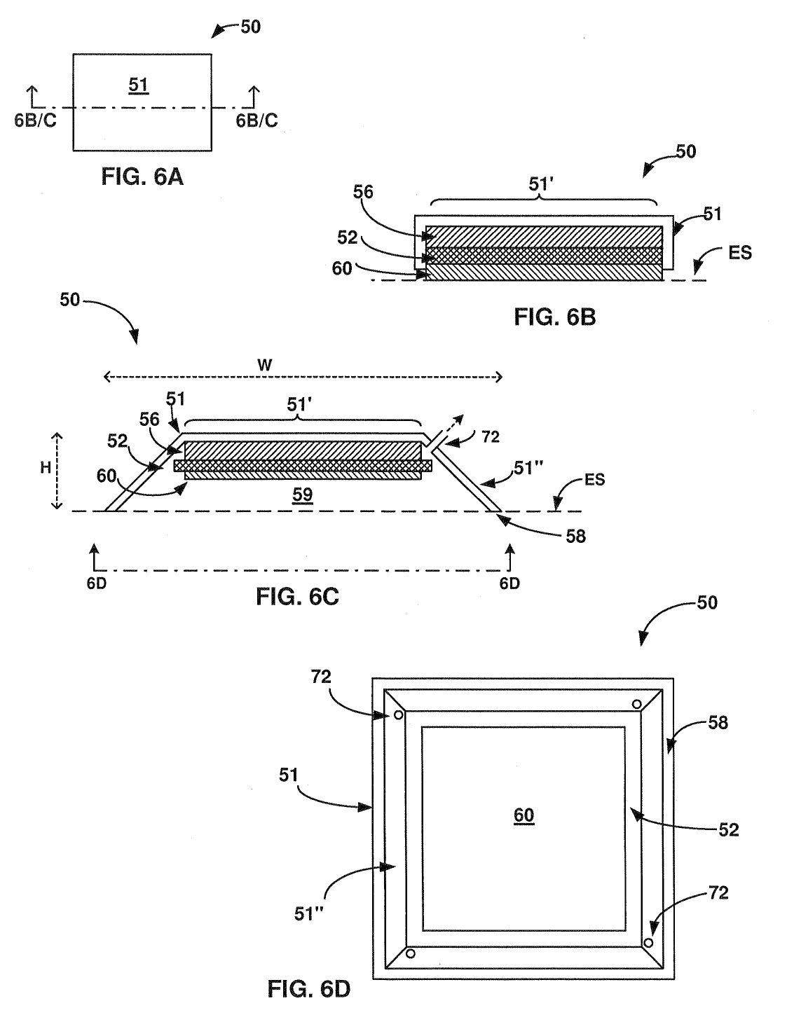

FIG. 4A is a block diagram schematically representing an electrosurgical system for treating a patient via first and second handpieces, according to another aspect of the invention. System 10 may include an electrosurgical generator 20, a first handpiece 50a, a second handpiece 50b, a user interface 26, and a vacuum unit 70. Each of first and second handpieces 50a, 50b may be configured for contacting an intact external surface of the skin on the patient's body or body part, PB/BP. Each of first and second handpieces 50a, 50b may be further configured for being affixed or adhered to the external surface of the skin of the patient, e.g., during a procedure of the instant invention in which adipose tissue disposed within or beneath the skin is to be targeted for ablation or other treatment. In an embodiment, each of first and second handpieces 50a, 50b may include at least one suction port 72 (see, e.g., FIG. 6D). Vacuum unit 70 may be in fluid communication with suction port(s) 72 of first and second handpieces 50a, 50b for providing suction sufficient to affix or adhere first and second handpieces 50a, 50b to the external skin surface during a procedure.

Electrosurgical generator 20 may be configured for providing a first AC voltage to first handpiece 50a and a second AC voltage to second handpiece 50b. User interface 26 may be coupled to, or in signal communication with, electrosurgical generator 20, for inputting thereto parameters related to a particular procedure. Such parameters may include the voltage and phase difference of first and second AC voltages provided to first and second handpieces 50a, 50b, as well as threshold temperature values for the target region of skin or target tissue. User interface 26 may also be coupled to, or in signal communication with, vacuum unit 70, for qualitatively and/or quantitatively controlling the application of suction, via vacuum unit 70, to first and second handpieces 50a, 50b.

FIG. 4B is a block diagram schematically representing an electrosurgical system including at least one temperature sensor 54, according to another aspect of the invention. System 10 may include an electrosurgical generator 20, a control unit 30, a first handpiece 50a, and a second handpiece 50b. First handpiece 50a may include a first electrode 60a, and second handpiece 50b may include a second electrode 60b. Each of first and second electrodes 60a, 60b may be at least substantially planar. Each of first and second electrodes 60a, 60b may be configured for contacting an external surface of the skin of a patient. In an embodiment, handpiece 50 may be configured for being affixed or adhered to an intact external skin surface of a patient.

Generator 20 may be configured for providing a first AC voltage and a second AC voltage to first and second handpieces 50a, 50b, respectively. The first and second AC voltages may be out of phase such that a potential difference exists between first and second electrodes 60a, 60b. Electric current flow between first and second electrodes 60a, 60b may provide electrical energy to a target tissue disposed between first and second handpieces 50a, 50b, wherein the electrical energy may be sufficient to remove or otherwise treat at least a portion of the target tissue. The target tissue may be at one or more regions of the patient's body, and first and second handpieces 50a, 50b may be separately manipulable to various skin locations such that each region of target tissue may be sequentially disposed between first and second electrodes 60a, 60b. The distribution of electric current between first and second electrodes 60a, 60b may be a function of the separation distance between first and second handpieces 50a, 50b (see, e.g., FIGS. 1A-B). Each of first and second handpieces 50a, 50b may be coupled to control unit 30. Control unit 30 may be integral with generator 20. The invention is not limited to any particular configuration for system 10.

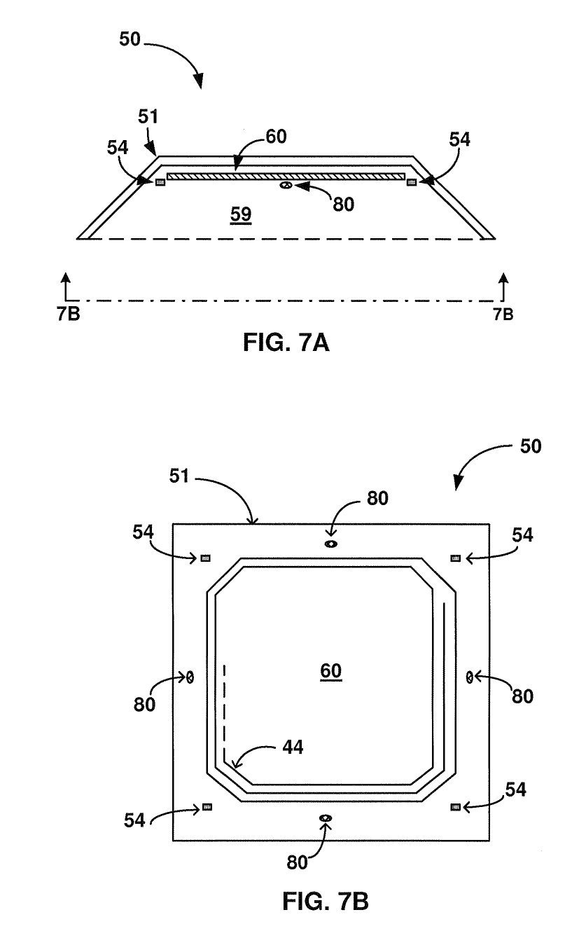

With further reference to FIG. 4B, each of first handpiece 50a and second handpiece 50b may further include at least one temperature sensor 54. Each temperature sensor 54 may be configured for contacting the skin of the patient. In an embodiment (not shown), one or more temperature sensors 54 may be disposed adjacent to first and second electrodes 60a, 60b, e.g., at the periphery, corners, or sides of each of first and second electrodes 60a, 60b (see, e.g., FIGS. 7A-B). Each temperature sensor 54 may be configured for independently sensing temperature values of a region of the patient's skin or other tissue in the vicinity of first and second electrodes 60a, 60b during treatment. First handpiece 50a may be configured for sensing temperature values at a first skin location, and second handpiece 50b may be configured for sensing temperature values at a second skin location (see, e.g., FIGS. 1A-B). In an embodiment, system 10 may be alternatively or additionally configured for sensing a temperature value of a target tissue, e.g., via extrapolation of a sensed skin temperature.

Each temperature sensor 54 may be in signal communication with control unit 30 for providing thereto sensed temperature values of the patient's skin or other tissue. Each of first and second electrodes 60a, 60b may also be in communication with control unit 30. Generator 20 may include an RF power source or supply (not shown in FIG. 4B) in communication with control unit 30. Control unit 30 may be configured for independently controlling power delivery to each of first and second electrodes 60a, 60b, e.g., in response to the skin or tissue temperature value(s) sensed by at least one temperature sensor 54 during a procedure. Control unit 30 may include an analog to digital converter in communication with a CPU, microprocessor, or microcontroller (not shown), and the like; however, the instant invention is not limited to a control unit 30 having particular components, circuitry, or configurations.

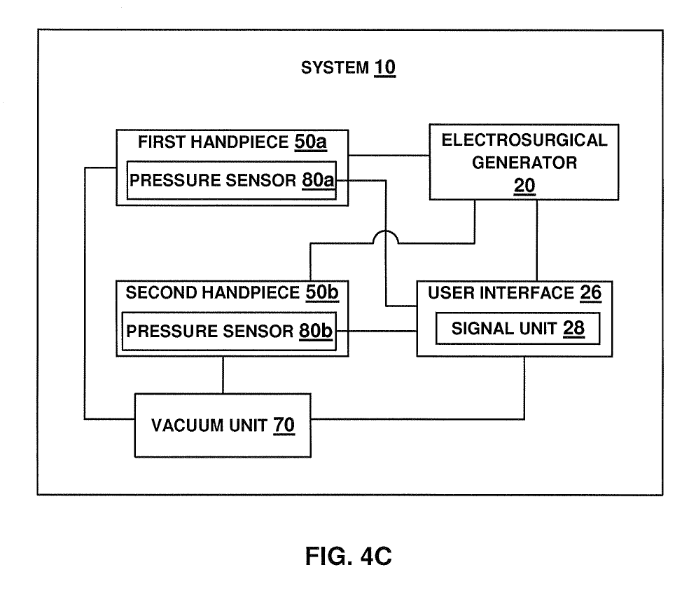

In other embodiments, the temperature of the treated skin or tissue may be controlled by controlling the voltage to a cooling unit 56 (see, e.g., FIGS. 5 and 6B-C). As an example, temperature sensor(s) 54 may be coupled to control unit 30, and the voltage supplied to cooling unit 56 may be controlled by control unit 30 in response to sensed skin/tissue temperature (see, e.g., FIGS. 7A-B). In an embodiment, cooling unit 56 may be integral with handpiece 50a/50b and may comprise a thermoelectric cooler (TEC).

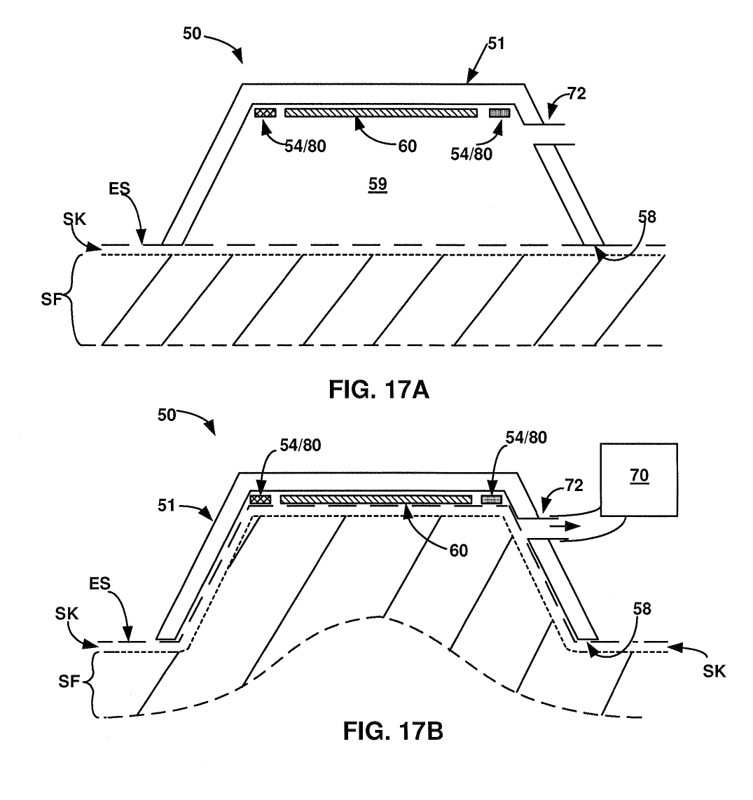

FIG. 4C is a block diagram schematically representing an electrosurgical system 10 for treating a patient via first and second handpieces, according to another aspect of the invention. System 10 may include an electrosurgical generator 20, first and second handpieces 50a, 50b coupled to generator 20, and a vacuum unit 70 in fluid communication with first and second handpieces 50a, 50b. First and second handpieces 50a, 50b may each include an electrode 60 (see, e.g., FIG. 5). During a procedure, suction provided by vacuum unit 70 may typically be sufficient to draw an area of the patient's skin towards first and second handpieces 50a, 50b, such that the external skin surface contacts electrode 60 (see, e.g., FIG. 17B).

System 10 may further include a user interface 26 coupled to vacuum unit 70 and to generator 20, substantially as described with reference to FIG. 4A. First and second handpieces 50a, 50b may further include a first pressure sensor 80a and a second pressure sensor 80b, respectively. Pressure sensors 80a/80b may also be referred to as pressure transducers, pressure senders, and the like. First and second handpieces 50a, 50b may be configured for monitoring patient contact therewith via first and second pressure sensors 80a, 80b, respectively. As an example, a fairly constant, low pressure sensed by pressure sensors 80a, 80b may indicate contact between electrodes 60a/60b and the patient's skin. Lack of contact between first electrode 60a or second electrode 60b and the patient may be indicated by an increase in pressure, e.g., above a threshold pressure value, as monitored by pressure sensors 80a and 80b, respectively. In an embodiment, pressure sensors 80a/80b may be disposed within a void of handpiece 50 (see, e.g., FIGS. 7A-B), such that pressure sensors 80a/80b are disposed in close proximity to the patient's tissue during a procedure.

With further reference to FIG. 4C, system 10 may further include a signal unit 28. System 10 may be configured for generating and/or emitting a warning signal via signal unit 28 (e.g., an audible and/or visual signal) in response to output from pressure sensors 80a/80b. Such a signal may serve to alert an operator (of system 10) of a relatively high pressure condition in the vicinity of pressure sensors 80a/80b, e.g., within void 59 of handpiece 50. As a non-limiting example, system 10 may be configured for generating a signal when the pressure sensed by pressure sensors 80a and 80b rises above a threshold pressure level, thereby indicating lack of patient-electrode contact. As shown in FIG. 4C, signal unit 28 may be integral with user interface 26; however, the skilled artisan will appreciate that alternative configurations are also within the scope of the invention.

In an embodiment, one or both of first and second handpieces 50a, 50b may include a plurality of pressure sensors (see, e.g., FIGS. 7A-B). In alternative configurations (not shown), pressure sensors 80a, 80b may be disposed elsewhere in the vacuum path between handpiece 50a/50b and vacuum unit 70. Pressure sensors 80a, 80b may be configured for sensing pressure at one or more locations along the vacuum path between vacuum unit 70 and shell 51. According to another aspect of the invention, contact of electrode 60 with the patient's skin may additionally or alternatively be monitored by sensing one or more electrical parameter(s), for example, electrode impedance.

### Electrosurgical Handpieces

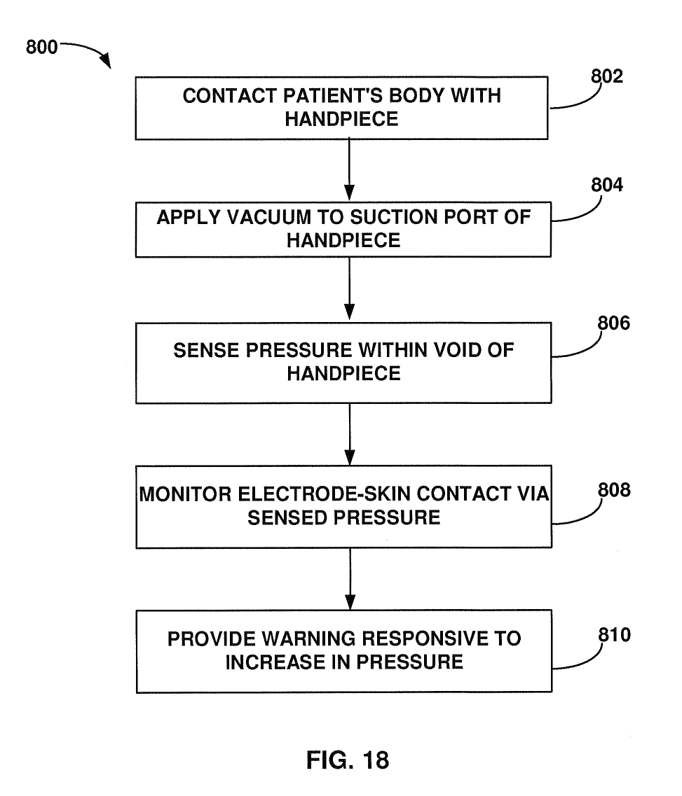

FIG. 5 is a block diagram schematically representing a handpiece, according to another embodiment of the invention. Handpiece 50a/50b may include a shell 51, a cooling unit 56, a support layer 52, and an electrode 60. Support layer 52 may be disposed between cooling unit 56 and electrode 60. Support layer 52 may comprise an electrically insulating and thermally conductive material. Electrode 60 may be in thermal communication with cooling unit 56 via support layer 52. Handpiece 50 may be configured for cooling the skin, via cooling unit 56, during a procedure. In an embodiment, cooling unit 56 may comprise a thermoelectric cooler (TEC). In an embodiment, skin temperature may be sensed by temperature sensor(s) 54 integral with handpiece 50a/50b (see, e.g., FIGS. 4A and 7A-B), and voltage supplied to cooling unit 56 may be adjusted according to sensed values of skin temperature, e.g., as described with reference to FIG. 18, infra.

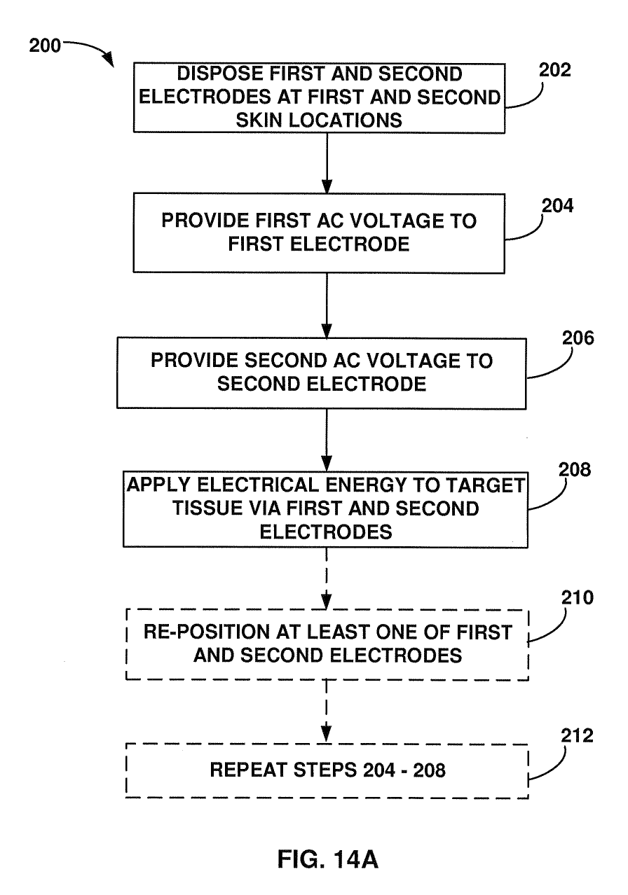

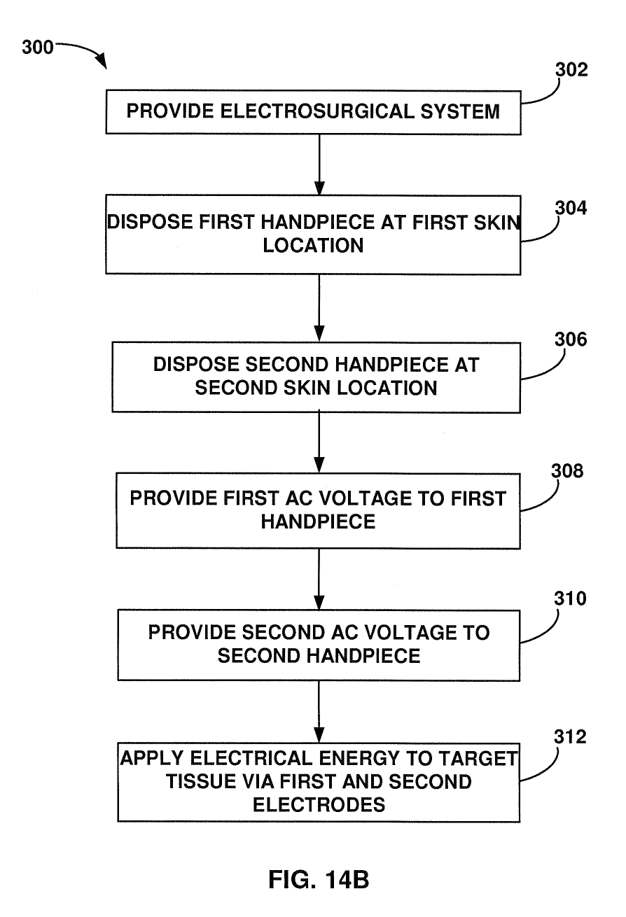

A handpiece having a cooling unit for cooling the skin or target tissue during a procedure is disclosed in commonly assigned, co-pending U.S. patent application Ser. No. 12/144,948, entitled “Subcutaneous Electric Field Distribution System and Methods,” (Atty. Docket No. ALTU-ALTU-3310), filed Jun. 24, 2008, the disclosure of which is incorporated by reference herein in its entirety. System 10 of the instant invention (see, e.g., FIGS. 1A-B, and 3A-4B) may typically include two separate handpieces, e.g., first and second handpieces 50a, 50b, both of which may be used concurrently and in the same or similar fashion to treat target tissue(s) (see, e.g., FIGS. 14A-B).

FIG. 6A is a schematic representation of a handpiece, in plan view as seen from above, according to another aspect of the invention. Handpiece 50 may include a shell 51. Shell 51 may comprise a rigid structure for housing an electrode 60 together with other elements or components of handpiece 50 (see, e.g., FIGS. 6B-D). In the embodiment of FIG. 6A, handpiece 50 is shown as having a substantially square or rectangular shape or outline. In some embodiments, handpiece 50 may have a substantially circular or round shape or outline. Naturally, other shapes or outlines for handpiece 50 are also within the scope of the invention. Handpiece 50 of FIGS. 6A-D may comprise first handpiece 50a or second handpiece 50b of system 10 (see, e.g., FIGS. 1A-B, and 3A-4B).

FIG. 6B is a sectional view of handpiece 50 of FIG. 6A, as seen along the line 6B/C-6B/C of FIG. 6A, according to an embodiment of the invention. Handpiece 50 may include a shell 51, an electrode 60, and a support layer 52. Shell 51 may be configured as a housing for supporting or protecting electrode 60. Electrode 60 may comprise first electrode 60a or second electrode 60b of system 10. Shell 51 may comprise an electrically insulating material, such as various plastics, and the like. Shell 51 may include a planar portion 51′, which may be substantially centrally located. Electrode 60 may be substantially planar. Electrode 60 may be disposed substantially centrally with respect to shell 51 and substantially parallel to planar portion 51′. In an embodiment, electrode 60 may comprise at least one spiral inductor 62 (see, e.g., FIGS. 9-12B). Handpiece 50 may include a cooling unit 56 in thermal communication with electrode 60. Cooling unit 56 may be disposed between shell 51 and support layer 52. Support layer 52 may be disposed between cooling unit 56 and electrode 60. Support layer 52 may comprise a thermally conductive and electrically insulating material, such as boron nitride. Electrode 60 may be in thermal communication with cooling unit 56 via support layer 52.

FIG. 6C is a sectional view of the handpiece of FIG. 6A, as seen along the line 6B/C-6B/C of FIG. 6A, according to another embodiment of the invention. Handpiece 50 of FIG. 6C may include an electrode 60, a support layer 52, a cooling unit 56, and a shell 51, substantially as described hereinabove with reference to handpiece 50 of FIG. 6B. Shell 51 may be configured for housing or supporting electrode 60 as well as other components of handpiece 50. Electrode 60 may be disposed substantially centrally within shell 51. In an embodiment as shown in FIG. 6C, shell 51 may include a collar portion 51″ extending distally from central planar portion 51′. Collar portion 51″ may terminate distally in a flange 58. Flange 58 may define a plane disposed substantially parallel to electrode 60 and/or shell planar portion 51′. Shell 51 may be substantially frusto-pyramidal or frusto-conical. In an embodiment, a void 59 within handpiece 50 may be configured for receiving skin or other tissue of the patient, and electrode 60 may be disposed apically within void 59.

With further reference to FIG. 6C, handpiece 50 may have a width, W and a height, H, wherein the width to height (W:H) ratio may typically be in the range of from about 5:1 to 15:1. Handpiece 50 may further include at least one suction port 72. Each suction port 72 may be coupled to a vacuum unit 70 (see, e.g., FIG. 4A). Each suction port 72 may be in fluid communication with void 59, and void 59 may be configured for applying suction to the external skin surface via vacuum unit 70. Handpiece 50 may be configured for contacting an area of the external surface of the skin, wherein the area is typically at least about 10 cm², usually at least about 20 cm², and often at least about 50 cm². In an embodiment, handpiece 50 may be configured for being affixed or adhered to an intact external skin surface of a patient. In an embodiment, handpiece 50 may be configured for sealing engagement of flange 58 against the external surface of the skin. In an embodiment, handpiece 50 may be affixed to the skin surface via suction applied to suction port(s) 72. In other embodiments, handpiece 50 may be affixed or adhered to the skin surface via an adhesive or a gel, and the like, wherein the adhesive, gel, or similar material may be applied to the skin surface and/or to one or more components of handpiece 50.

FIG. 6D is a plan view of the handpiece of FIG. 6C, as seen along the line 6D-6D of FIG. 6C. As shown in FIG. 6D, electrode 60 may be disposed at a substantially central location with respect to the distal perimeter of handpiece 50 as defined by flange 58. In an embodiment, electrode 60 may comprise a spiral inductor (see, e.g., FIGS. 9-12B). Suction ports 72 may be disposed peripherally around electrode 60, e.g., within collar portion 51″. Although four suction ports 72 are shown in FIG. 6D, other numbers and arrangements of suction ports 72 are within the scope of the invention. Similarly, although, handpiece 50 and electrode 60 are shown in FIG. 6D as being substantially square in outline, other shapes are also possible under the invention.

FIG. 7A is a sectional side view of a handpiece including at least one pressure sensor, according to another embodiment of the invention. Handpiece 50 may include a shell 51, an electrode 60, at least one temperature sensor 54, and at least one pressure sensor 80. Each pressure sensor 80 may be configured for sensing pressure within shell 51 of handpiece 50. Shell 51 may be substantially frusto-conical, frusto-pyramidal, or dome-shaped. Electrode 60 may be at least substantially planar and disposed within a void 59 defined by shell 51.

FIG. 7B schematically represents handpiece 50 of FIG. 7A, as seen in plan view along the line 7B-7B of FIG. 7A. As shown, pressure sensors 80 and temperature sensors 54 may be disposed adjacent to the periphery, corners, or sides of electrode 60, i.e., within void 59. Void 59 within handpiece 50 may be configured for receiving an area or region of skin or underlying tissue during a procedure. In an embodiment, the skin received by void 59 may be adjacent to a target tissue to be treated by handpiece 50. Although FIG. 7B shows four (4) pressure sensors 80 and four (4) temperature sensors 54, each spaced around the periphery of electrode 60, it will be apparent to the skilled artisan that alternative numbers and arrangements of both pressure sensors 80 and temperature sensors 54 are also within the scope of the invention. As shown, electrode 60 may comprise a spiral inductor 44, however the invention is by no means limited to a spiral electrode configuration.

FIG. 8A schematically represents an electrosurgical system 10 according to another embodiment of the invention. System 10 may include a first electrode-bearing pad 50a′, a second electrode-bearing pad 50b′, and an electrosurgical generator 20. First and second electrode-bearing pads 50a′, 50b′ may be separately coupled to electrosurgical generator 20, e.g., substantially as described with reference to FIG. 1A.

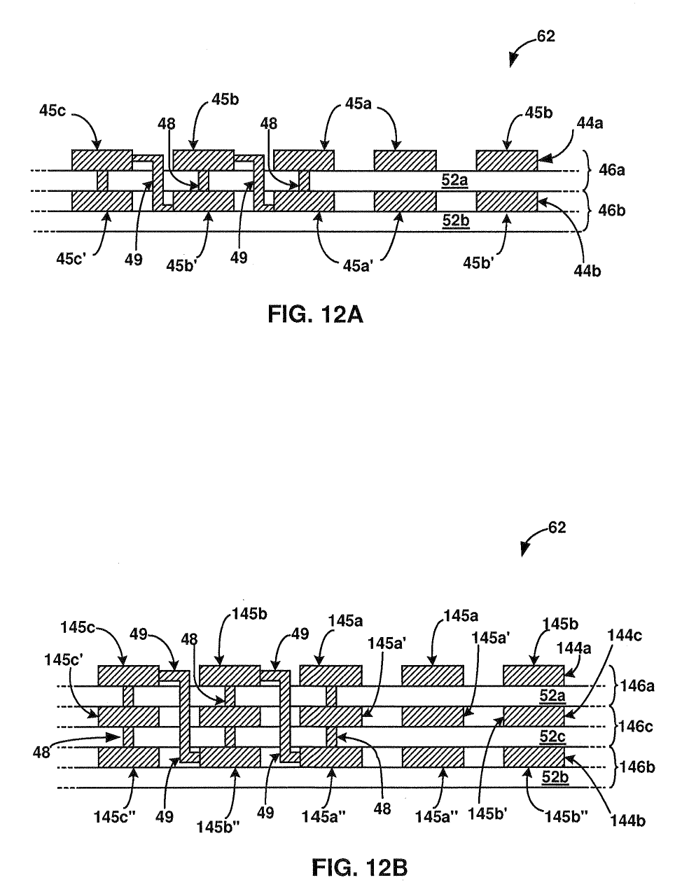

In FIG. 8A, first and second electrode-bearing pads 50a′, 50b′ are schematically represented as seen in side view. First and second electrode bearing pads 50a′, 50b′ may each include an electrode 60a, 60b, respectively. In an embodiment, one or both of electrodes 60a, 60b may comprise a spiral inductor 44 (see, e.g., FIGS. 11 and 12A-B). Each electrode 60a, 60b may be disposed on a support layer 52a, 52b, respectively. In an embodiment, support layer 52a, 52b may comprise a flexible, electrically insulating sheet. Electrosurgical generator 20 may be configured for concurrently providing a first AC voltage to first electrode-bearing pad 50a′ and a second AC voltage to second electrode-bearing pad 50b′, essentially as described with reference to FIG. 1A. The first and second AC voltages may be about 180° out of phase (see, e.g., FIG. 2B).

Each of first and second electrode-bearing pads 50a′ and 50b′ may be configured for contacting an area of an intact external surface of skin of the patient's body or body part, substantially as described with reference to FIG. 1A. In an embodiment, electrode-bearing pads 50a′, 50b′ may be of substantially the same size and configuration. In another embodiment, the electrode-bearing pads 50a′, 50b′ may be of different size and/or shape. As an example, the size and/or shape of first and second electrode-bearing pads 50a′ and 50b′ may be varied according to various region(s) of the patient's body to be treated.

Each of first and second electrode-bearing pads 50a′ and 50b′ may be separately or independently movable with respect to each other, and each may be disposed at various selected locations on the patient's body, substantially as described with reference to FIG. 1A. In an embodiment, electrode-bearing pads 50a′ and 50b′ may be flexible and/or conformable to the contour of a portion of the patient's body to be treated (see, e.g., FIG. 8B).

First and second electrode-bearing pads 50a′ and 50b′ may be configured for being affixed or adhered to an external skin surface of the patient. In an embodiment, each of first and second electrode-bearing pads 50a′, 50b′ may further comprise a patient-contacting layer 61, which may be disposed on electrodes 60a, 60b. In an embodiment, patient-contacting layer 61 may comprise an adhesive that promotes adherence of pads 50a′, 50b′ to the patient's skin. In an embodiment, patient-contacting layer 61 may comprise an electrically conductive material having an electrical resistivity value less than 0.1 Ohm·m, and in some embodiments 0.01 Ohm·m or less.

FIG. 8B schematically represents a conformable electrode-bearing pad 50a′/50b′, as seen in side view in relation to a portion of a patient's body, according to another embodiment of the invention. Electrode-bearing pad 50a′/50b′ may be flexible, and may be conformed to the contour of a portion of the patient's body, PB, or body part, BP, to be treated. As a non-limiting example, pad 50a′/50b′ may include electrode 60a/60b disposed on support layer 52a/52b (see, e.g., FIG. 8A), wherein both electrode 60a/60b and support layer 52a/52b may be flexible and conformable to the patient's body. Electrode 60a/60b and support layer 52a/52b are not shown in FIG. 8B for the sake of clarity.

### Spiral Inductors

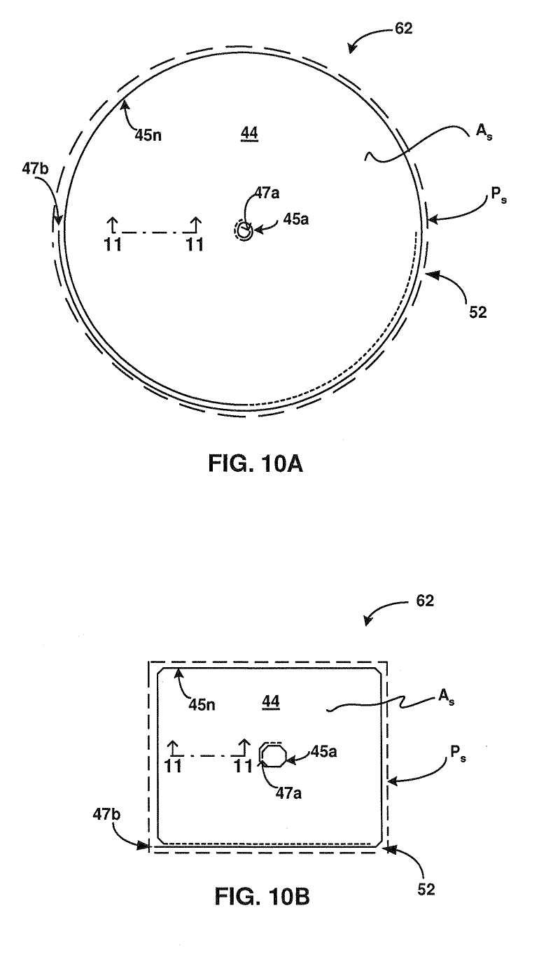

FIG. 9 schematically represents a spiral of electrically conductive material for forming an electrode, as seen in plan view, according to another embodiment of the invention. Spiral 44 may include a plurality of turns 45 and an inner terminus 47a. Inner terminus 47a may be coupled to a feedpoint (not shown) for coupling spiral 44 to an electrosurgical power supply or generator. In an embodiment, one or more spirals 44 may form a spiral inductor 62 (see, e.g., FIGS. 10A-12B). Each of first and second electrodes 60a and 60b of first and second handpieces 50a, 50b may comprise spiral inductor 62 (see, e.g., FIG. 13). Although spiral 44 of FIG. 9 is shown as substantially round, other configurations are also within the scope of the invention (see, e.g., FIG. 10B).

Spiral 44 may comprise a spiral trace of an electrically conductive metal, such as Cu, Al, or various alloys, as non-limiting examples. In an embodiment, spiral 44 may comprise a filament of the electrically conductive metal, wherein the filament may be disposed on a support layer 52 (see, e.g., FIGS. 810A-11). Only a few of the radially inner turns 45 of spiral 44 are shown in FIG. 9, whereas spiral 44 in its entirety may comprise from about 10 to 200 or more turns, typically from about 10 to 150 turns, and often from about 15 to 100 turns.

As shown in FIG. 9, spiral 44 may have a pitch, Pₜ, representing a radial distance between the radial midpoints of adjacent turns 45. The pitch of spiral 44 may be in the range of from about 0.1 mm to 10 mm or more, typically from about 0.2 mm to 9 mm, often from about 0.25 to 5 mm, and in some embodiments from about 0.3 to 1.5 mm. In an embodiment, the pitch of spiral 44 may be constant or substantially constant. In other embodiments, the pitch of spiral 44 may vary.

Turns 45 of spiral 44 may have a width, Wₜ, wherein the width, Wₜ is a radial distance across each turn 45. The width of each of turns 45 may typically be in the range of from about 0.05 mm to 10 mm or more, typically from about 0.15 to 9 mm, often from about 0.2 to 5 mm, and in some embodiments from about 0.25 to 1.5 mm. In an embodiment, the width of the various turns 45 may be constant or substantially constant. In other embodiments, the width of turns 45 may vary. A profile or cross-sectional shape of turns 45 may be substantially rectangular or rounded. Typically, the width of each turn 45 may be greater than its height.

A gap, G may exist between adjacent turns 45 of spiral 44, wherein the gap may represent a radial distance between opposing edges of adjacent turns 45. The gap is typically much less than the pitch. The gap is typically much less than the width, usually the gap is substantially less than the width, and often the gap is considerably less than the width. In an embodiment, the width, Wₜ may typically be at least twice as great as the gap, G (Wₜ≧2*G). In some embodiments, the width may typically be from three (3) to 15 times (3×-20×) the gap.

The gap between turns 45 of spiral 44 may typically be in the range of from about 0.1 mm to 0.5 mm, usually from about 0.15 to 0.4 mm, and often from about 0.15 to 0.3 mm. In an embodiment, the gap between adjacent turns 45 may be constant or substantially constant, even though the pitch may be variable. Spirals of electrically conductive material suitable for forming spiral inductors are disclosed in commonly assigned, co-pending U.S. patent application Ser. No. 11/966,895, entitled “High Conductivity Inductively Equalized Electrodes and Methods,” (Atty. Docket No. ALTU-3000), filed Dec. 28, 2007, the disclosure of which is incorporated by reference herein in its entirety.

FIG. 10A schematically represents a spiral inductor, as seen in plan view, according to an embodiment of the invention. Spiral inductor 62 may be used to form first and second electrodes 60a, 60b of first and second handpieces 50a, 50b. Spiral inductor 62 of FIG. 10A may have a substantially circular or oval configuration. Spiral inductor 62 may include a spiral trace 44 of electrically conductive metal including an inner terminus 47a and an outer terminus 47b. In an embodiment, spiral inductor 62 may further include a support layer 52, wherein spiral 44 may be disposed on support layer 52 (see, e.g., FIG. 11). In an embodiment, support layer 52 may comprise an electrically insulating or dielectric material. In an embodiment, support layer 52 may comprise a material that is both thermally conductive and electrically insulating.

Spiral inductor 62 may include a plurality of turns, from a first turn 45a (radially innermost) to an n^{th} turn 45n (radially outermost). In an embodiment, n may be from about 10 to 200 or more, substantially as described hereinabove. Spiral inductor 62 may have a perimeter, Pₛ, and an external surface area, Aₛ, defined by the perimeter. The electrically conductive metal of spiral 44 may occupy at least about 50% of a total surface area Aₛ, that is to say, at least about 50 percent (%) of the external surface area of spiral inductor 62 may be occupied by the electrically conductive metal of spiral 44. Typically, electrically conductive metal of spiral 44 may occupy from about 60 to 99% of external surface area, Aₛ; usually from about 70 to 99% of external surface area, Aₛ; often from about 75 to 98% of external surface area, Aₛ; and in some embodiments electrically conductive metal of spiral 44 may occupy from about 85% to 97% of external surface area, Aₛ.

FIG. 10B schematically represents a spiral inductor 62, as seen in plan view, according to another embodiment of the invention. Each of first and second electrodes 60a, 60b may comprise spiral inductor 62. Spiral inductor 62 may include a spiral trace 44 of electrically conductive metal having an inner terminus 47a, an outer terminus 47b, and a plurality of turns, 45a-n, substantially as described for the embodiment of FIG. 10A. Spiral inductor 62 of FIG. 10B may have a substantially square or rectangular configuration, a perimeter, Pₛ, and a surface area Aₛ defined by the perimeter. Spiral inductor 62 may include a spiral trace 44 of electrically conductive metal. Spiral trace 44 may occupy a percentage of surface area, Aₛ generally as described with reference to FIG. 10A.

It is to be understood that spiral inductor 62 is not limited to a substantially round or rectangular configuration as shown in FIGS. 10A-B, respectively; instead other shapes for spiral inductor 62 are also within the scope of the invention. In an embodiment, spiral inductors 62 of FIGS. 10A-B may comprise a spiral 44 which may be at least substantially planar.

FIG. 11 is a sectional view of a portion of the spiral inductor of FIGS. 10A-B, as seen along the line 11-11 of FIGS. 10A-B, according to an embodiment of the invention. FIG. 11 shows spiral inductor 62 in relation to the external surface of the patient's skin, ES. (Note that FIG. 11 shows spiral inductor 62 as being inverted in comparison with the orientation shown in FIGS. 10A-B). In an embodiment, spiral inductor 62 may be used as an electrode 60a/60b for handpieces 50a and 50b. As shown in FIG. 1, spiral inductor 62 may be at least substantially planar.

With further reference to FIG. 11, spiral inductor 62 may comprise a spiral 44 of electrically conductive metal. In an embodiment, spiral inductor 62 may further comprise a support layer 52, wherein spiral 44 may be disposed on support layer 52. In an embodiment, support layer 52 may be disposed in thermal communication with cooling unit 56 of handpiece 50 (see, e.g., FIGS. 6B-C). In an embodiment, support layer 52 may comprise an electrically insulating and thermally conductive material. In another embodiment, support layer 52 may comprise a layer of electrically insulating adhesive.

Spiral inductor 62 may include an external surface 66 for contacting the patient, e.g., the patient's external skin surface. In an embodiment, spiral inductor 62 may further comprise a patient-contacting layer 61, which may be disposed on the metal surface of spiral 44, such that patient-contacting layer 61 comprises external surface 66. The patient-contacting layer may comprise an electrically conductive material having an electrical resistivity value less than 0.1 Ohm·m, and in some embodiments 0.01 Ohm·m or less. In another embodiment, patient-contacting layer 61 may be omitted, whereby external surface 66 may be a bare metal surface of electrically conductive metal spiral 44.

FIG. 12A is a schematic sectional view of a spiral inductor having a plurality of spirals, showing electrical connections between each spiral, according to another aspect of the invention. Spiral inductor 62 of FIG. 12A may include a first or outermost spiral 44a and a second or innermost spiral 44b. Spirals 44a and 44b may be disposed on a first or outermost support layer 52a and an innermost support layer 52b, respectively. Spiral inductor 62 may be a component of an electrode 60 for handpieces 50a, 50b (see, e.g., FIGS. 5 and 13). An external surface of first or outermost spiral 44a may contact the skin of a patient during a procedure involving handpieces 50a, 50b. First and second spirals 44a, 44b may each have a plurality of turns, only three of which are shown in FIG. 12A for the sake of clarity. Each of the plurality of turns of first and second spirals 44a, 44b, including those specifically shown in FIG. 12A, as well as additional turns not shown in FIG. 12A, may be referred to herein generically as “turns 45” (see, e.g., FIGS. 9 and 10A-B). In an embodiment, first and second spirals 44a, 44b may each have the same number of turns 45. In another embodiment, the number of turns of first and second spirals 44a, 44b may be different; for example, first spiral 44a may have one or more additional turns as compared with second spiral 44b, or vice versa. In an embodiment, first and second spirals 44a, 44b may each have the same pitch (see, e.g., FIG. 9).

As shown in FIG. 12A, radially corresponding turns of first and second spirals 44a, 44b may be interconnected by vertical connections 48, while connection between turns of second spiral 44b and adjacent radially outward turns of first spiral 44a (i.e., between radially non-corresponding turns) may be by radial connections 49. Vertical connections 48 and/or radial connections 49 may be referred to generally herein as “vias.”

First and second spirals 44a, 44b may be aligned or stacked such that the first turn 45a of first spiral 44a may be vertically aligned with first turn 45a′ of second spiral 44b, as shown in FIG. 12A. In the spiral inductor 62 of FIG. 12A, turns 45 of spirals 44a, 44b may be interconnected between spiral layers 46a and 46b as follows:

1) first turn 45a of first spiral 44a may be electrically coupled to a first turn 45a′ of second spiral 44b,

2) first turn 45a′ of second spiral 44b may be electrically coupled to a second turn 45b of first spiral 44a,

3) second turn 45b of first spiral 44a may be electrically coupled to a second turn 45b′ of second spiral 44b,

4) second turn 45b′ of second spiral 44b may be electrically coupled to a third turn 45c of first spiral 44a, and

5) third turn 45c of first spiral 44a may be electrically coupled to a third turn 45c′ of second spiral 44b. This same pattern or sequence of interconnection may be continued for all successive turns (not shown in FIG. 12A) of first and second spirals 44a, 44b.

It is to be understood that the coupling between specific turns enumerated hereinabove may be performed in sequences other than as listed to provide a multi-layer spiral inductor having turns 45 electrically coupled as shown in FIG. 12A. The manner of electrical coupling of first and second spirals 44a, 44b as shown in FIG. 12A may be summarized in more general terms as follows:

i) each turn of first spiral 44a may be electrically coupled to a radially corresponding turn of second spiral 44b, and

ii) each turn of second spiral 44b may be electrically coupled to an adjacent radially outward turn of first spiral 44a. However, interconnection of first and second spirals 44a, 44b according to item ii) may be governed by the proviso that, if the number of turns of second spiral 44b is equal to or greater than the number of turns of first spiral 44a, the radially outermost turn of second spiral 44b will lack an adjacent radially outward turn on first spiral 44a; in which case interconnection of spirals 44a, 44b may terminate at the radially outermost turn of second spiral 44b. (Or, in a description of electrical coupling between spirals 44a, 44b that proceeds in a radially inward direction (as opposed to radially outward, as described above), interconnection of spirals 44a, 44b may be said to begin at the radially outermost turn of second spiral 44b to provide spiral inductor 62 of FIG. 12A.) In alternative embodiments, a plurality of stacked spirals 44, which may have identical or non-identical spiral configurations, e.g., different numbers of turns 45, may be electrically interconnected generally as shown in FIG. 12A to form spiral inductors 62 which are also within the scope of the invention.

For purposes of illustration, FIG. 12A shows only three turns of each spiral 44a, 44b, e.g., first, second, and third turns 45a, 45b, 45c, respectively, of first spiral 44a. In practice, each spiral 44a, 44b may comprise from about 10 to 200 turns, typically from about 20 to 150 turns, often from about 30 to 150 turns, and usually from about 40 to 120 turns. However, the manner of interconnecting turns 45 of spirals 44a, 44b may be as shown in FIG. 12A regardless of the number of turns in each spiral.

FIG. 12B is a schematic sectional view of a multi-layer spiral inductor 62, showing electrical connections between each of a plurality of spirals 144a-c, according to another embodiment of the invention. Each spiral 144a-c may comprise a spiral trace of electrically conductive metal, and the plurality of spirals 144a-c may be vertically stacked or aligned and electrically interconnected as shown. Spiral inductor 62 may be a component of an electrode 60a, 60b for handpieces 50a, 50b. Spiral inductor 62 may include a first or outermost spiral layer 146a and an innermost spiral layer 146b, wherein first or outermost spiral layer 146a may contact the external surface of the skin of a patient during a procedure. Spiral inductor 62 may further include at least one intermediate spiral layer 146c.

For clarity of illustration, only a central portion of spiral inductor 62 with a single intermediate spiral layer is shown in FIG. 12B, it being understood that spiral inductor 62 may comprise a plurality of intermediate spiral layers, and that each spiral 144a-c may comprise up to 200 or more turns, e.g., as described with reference to FIG. 12A. In the description of FIG. 12B, the turns of each spiral 144a-c may be referred to non-specifically as turns 45 (see, e.g., FIG. 9).

Each spiral 144a-c may comprise an electrically conductive metal, for example as a metal trace or filament. In an embodiment, spirals 144a-c may each have the same spiral configuration, e.g., each spiral 144a-c may have the same number of turns, the same pitch, the same trace width, and the same gap between adjacent turns (see, e.g., FIG. 9). In an embodiment, spirals 144a-c may be stacked such that radially corresponding turns of each of spirals 144a, 144b, and 144c are vertically aligned with each other. Spirals 144a, 144b, and 144c may be disposed on a first or outermost support layer 52a, an innermost support layer 52b, and an intermediate support layer 52c, respectively.

With still further reference to FIG. 12B, turns 45 of spirals 144a, 144b, and 144c may be electrically coupled in the following manner:

I) each turn of outermost spiral 144a may be electrically coupled to a radially corresponding turn of each successive spiral 144c and 144b, i.e., first turn 145a of first spiral 144a may be coupled to first turn 145a′ of intermediate spiral 144c, which may be coupled to first turn 145a″ of innermost spiral 144b, and

II) each turn of innermost spiral 144b may be electrically coupled to an adjacent, radially outward turn of outermost spiral 144a, e.g., first turn 145a″ of spiral 144b may be electrically coupled to second turn 145b of spiral 144a. This same pattern or sequence of interconnection may be continued for all successive turns (not shown in FIG. 12B) of spirals 144a-c. An exception to connection according to item II) (analogous to the proviso described with reference to FIG. 12A) may exist for the radially outermost turn of innermost spiral 144b if the number of turns of innermost spiral 144b is equal to or greater than the number of turns of outermost spiral 144a.

The same manner of interconnection as described with reference to FIG. 12B may be used for electrically interconnecting multi-layer spiral inductors 62 having larger numbers of spiral layers and/or larger numbers of turns 45. Each turn 45 may be electrically coupled, in series, to a radially corresponding turn of each successive spiral by vertical connections 48, while each turn 45 of innermost spiral 144b may be electrically coupled to an adjacent, radially outward turn of outermost spiral 144a by radial connections 49. In an embodiment, a plurality of vertical connections 48, which couple a plurality of radially corresponding turns of successive spirals 144a-c, may be vertically aligned. Vertical connections 48 and/or radial connections 49 may be referred to generally herein as “vias.”

FIG. 13 is a sectional view of a handpiece including an electrode comprising a spiral inductor having a plurality of vertically aligned spirals, according to another embodiment of the invention. In FIG. 13 handpiece 50a/50b is shown in relation to the external surface of the patient's skin, ES. Handpiece 50a/50b may be configured for contacting the external surface of the skin. Spiral inductor 62 may include an external surface 66 for contacting the patient, e.g., the patient's external skin surface, during a procedure for treating the skin or subcutaneous fat. The term “vertical” as used herein may refer to a direction at least substantially orthogonal to external surface 66 of spiral inductor 62.

Handpiece 50a/50b may include a shell 51, a cooling unit 56, an inner support layer 52b, an outer support layer 52a, and first and second spirals 44a, 44b, respectively. At least a portion of each of shell 51, cooling unit 56, inner support layer 52b, outer support layer 52a, and first and second spirals 44a, 44b may be substantially planar. Cooling unit 56 may be disposed adjacent to shell 51. Cooling unit 56 may have elements and features as described hereinabove with respect to other embodiments of the invention. First spiral 44a may be disposed on outer support layer 52a, and second spiral 44b may be disposed on inner support layer 52b. Each of first and second spirals 44a, 44b may comprise a spiral of electrically conductive metal. First and second spirals 44a, 44b may be interconnected, e.g., as described with reference to FIG. 12A, to form a spiral inductor 62, wherein spiral inductor 62 may function as an electrode 60 of handpiece 50a/50b. According to an aspect of the present invention, two (e.g., a pair of) handpieces 50a and 50b (see, e.g., FIGS. 1A-B, 4A) may be used concurrently and in combination for treating a single target tissue of the patient.

With further reference to FIG. 13, outer support layer 52a may comprise an electrically insulating material. In an embodiment, outer support layer 52a may comprise a polyester film or sheet, such as Mylar® (DuPont Teijin Films). Inner support layer 52b may comprise a thermally conductive and electrically insulating material. In an embodiment, inner support layer 52b may comprise boron nitride. Cooling unit 56 may be in thermal communication with electrode 60 via inner support layer 52b. Inner support layer 52b may be disposed on or adjacent to cooling unit 56.

### Methods for Treating a Patient

FIG. 14A is a flow chart schematically representing steps in a method 200 for treating a patient, according to another embodiment of the invention. As a non-limiting example, method 200 may be used in the treatment of targeted subcutaneous fat, for body sculpting procedures, treating cellulite, and the like. Step 202 may involve disposing a first electrode and a second electrode at a first location and a second location, respectively, on the patient's skin. (The first and second locations on the skin may be referred to as the first and second skin locations, respectively.) The target tissue may be disposed generally within a zone of electric current distribution between the first and second electrodes (see, e.g., FIGS. 1A-B). As non-limiting examples, one or both of the first and second skin locations may be on or at a part of the patient's body such as the abdomen, the back, the buttocks, the hips, the thighs, the upper arms, or the neck.

Each of the first and second electrodes may be electrically coupled to an electrosurgical generator configured for providing a first AC voltage and a second AC voltage to the first and second electrodes, respectively; and each of the first and second electrodes may deliver electrical energy to the target tissue. Furthermore, the first and second electrodes may be independently movable, or separately manipulable, with respect to each other. In an embodiment, the first and second electrodes may have at least substantially the same components, configuration, and structure. In an embodiment, each of the first and second electrodes may comprise a spiral inductor.

Step 204 may involve providing a first AC voltage to the first electrode, and step 206 may involve providing a second AC voltage to the second electrode, wherein steps 204 and 206 may be performed concurrently. In an embodiment, the first and second AC voltages may be about 180° out of phase. Step 208 may involve applying electrical energy to the target tissue via the first and second AC voltages provided to the first and second electrodes. Before or during steps 204-208, parameters of the first and second AC voltages, such as frequency and degree of phase difference, may be selected or adjusted via a user interface coupled to the power supply. The electrical energy applied in step 208 may typically be sufficient to remove or modify at least a portion of the target tissue, whereby the shape of the patient's body in the treated region may sculpted, and/or the appearance of cellulite on the skin may be decreased.

Optionally, step 210 may involve re-positioning at least one of the first and second electrodes, either locally to re-treat the target tissue, or to a different region of the patient's body to treat additional target tissue. Thereafter, step 212 may involve repeating steps 204 through 208.

FIG. 14B is a flow chart schematically representing steps in a method 300 for treating a patient, according to another embodiment of the invention. As a non-limiting example, method 300 may be used in the treatment of target tissues such as subcutaneous fat, and the like. Step 302 may involve providing an electrosurgical system having first and second handpieces, e.g., a pair of similarly constructed if not substantially identical handpieces, wherein the two handpieces may be separately manipulable and configured for contacting the skin of a patient at various selected locations on the patient's body. The first and second handpieces may include a first electrode and a second electrode, respectively. The first handpiece may include a first cooling unit in thermal communication with the first electrode, and the second handpiece may include a second cooling unit in thermal communication with the second electrode. In an embodiment, each of the first and second electrodes may comprise a substantially planar spiral inductor, wherein each spiral inductor may comprise one or more spirals of electrically conductive metal.

Each of the first and second electrodes may be configured for contacting an intact external surface of the skin. By “intact” skin surface is meant a skin surface that does not have any significant lacerations, surgical incisions, or the like. The external surface of the skin may have one or more scars, wrinkles, discolorations, blemishes, pimples, and/or other surface imperfections, including cellulite.

The system provided in step 302 may include an electrosurgical generator configured for providing a first AC voltage to the first electrode and a second AC voltage to the second electrode, wherein the first and second AC voltages may be 180° out of phase.

Steps 304 and 306 may involve disposing the first and second handpieces at a first skin location and a second skin location, respectively, such that an external surface of each of the first and second electrodes contacts the external surface of the patient's skin at the respective first and second skin locations. A target tissue, such as a layer or pocket of subcutaneous fat, may be disposed generally within a zone of electric current distribution between the first and second electrodes.

Method 300 may be applicable to the treatment of numerous different regions of a patient's body. For example, the first and second skin locations may be on one or more of the following parts of the body: the abdomen, the back, the buttocks, the hips, the thighs, the upper arms, and the neck. In an embodiment, the first skin location may be on the abdomen, and the second skin location may be on the lower back of the patient. In another embodiment, the first skin location may be on a first buttock or a second buttock (e.g., the right or left buttock), and the second skin location may be on the first buttock or the second buttock of the patient. Stated differently, the first and second skin locations may be on the same buttock (left or right), or the first and second skin locations may be on opposite buttocks. In another embodiment, the first skin location may be on a first part of the thigh, and the second skin location may be on a second part of the same thigh. As a non-limiting example, the first skin location may be on the outside of the thigh, and the second skin location may be on the inside of the thigh. In another embodiment, the first skin location may be on a first part of the arm, and the second skin location may be on a second part of the same arm. As a non-limiting example, the first skin location may be on the anterior of the arm, and the second skin location may be on the posterior of the arm.

In an embodiment, the first and second handpieces may have the same or similar size and structure, and the first and second handpieces may be used interchangeably at the first and second skin locations. The invention is not limited to any particular part of the body, nor to those parts of the body listed herein. Parts of the body to be treated according to the instant invention, as well as the quality and quantity of treatment, may vary widely from patient to patient.

Step 308 may involve providing a first AC voltage to the first electrode, and step 310 may involve providing a second AC voltage to the second electrode, wherein steps 308 and 310 may be performed concurrently. In an embodiment, the first and second AC voltages may be of approximately equal magnitude and opposite polarity, providing a potential difference between the first and second electrodes.

Step 312 may involve applying electrical energy to the target tissue via the first and second AC voltages provided to the first and second electrodes. The electrosurgical system provided in step 302 may include a user interface coupled to the power supply, by which an operator may select various treatment parameters before or during a procedure according to method 300. For example, before or during steps 308-312, parameters of the first and second AC voltages, such as frequency and degree of phase difference, may be selected or adjusted via the user interface. The electrical energy applied in step 312 may typically be sufficient to remove or modify at least a portion of the target adipose tissue, whereby the appearance of the patient's body and/or skin may be improved.

In an embodiment, the first and second handpieces may each include a suction port, and the system provided in step 302 may further include a vacuum unit coupled to the first and second handpieces via their respective suction ports. The first and second handpieces may be affixed to the patient's skin, e.g., during steps 308-312, via suction applied to the first and second handpieces by the vacuum unit.

Optionally, one or both of the first and second electrodes may be re-positioned to a different skin location on the patient's body, and thereafter steps 308-312 may be repeated. For example, the distribution of the electric field within the patient's tissues may be varied by changing the separation distance between the first and second handpieces, thereby allowing treatment of a target tissue at a particular location or depth beneath the skin. As noted hereinabove, numerous different regions of the body may be treated according to method 300.

In an embodiment, various methods of the instant invention may similarly use an electrode-bearing pad (see, e.g., FIGS. 17A-B) in lieu of an electrosurgical handpiece per se.

### Methods for Making Electrosurgical Handpieces

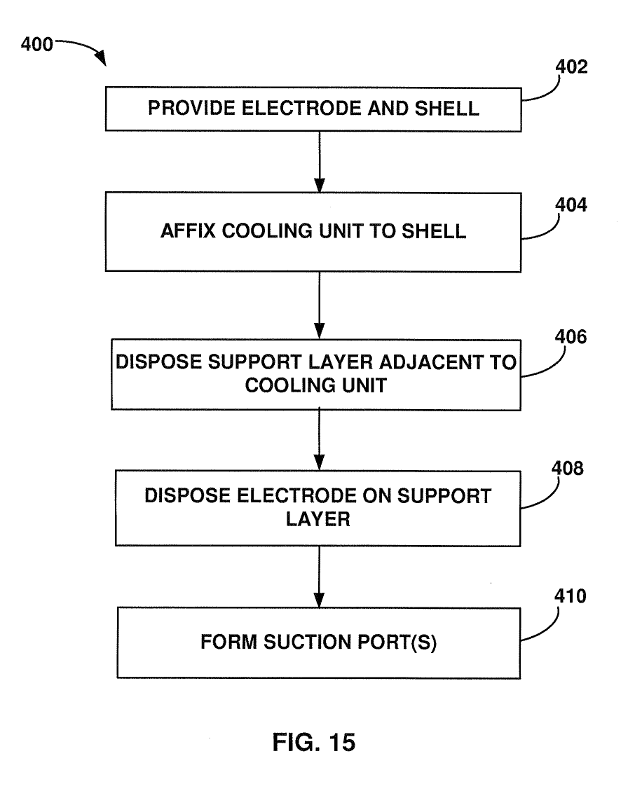

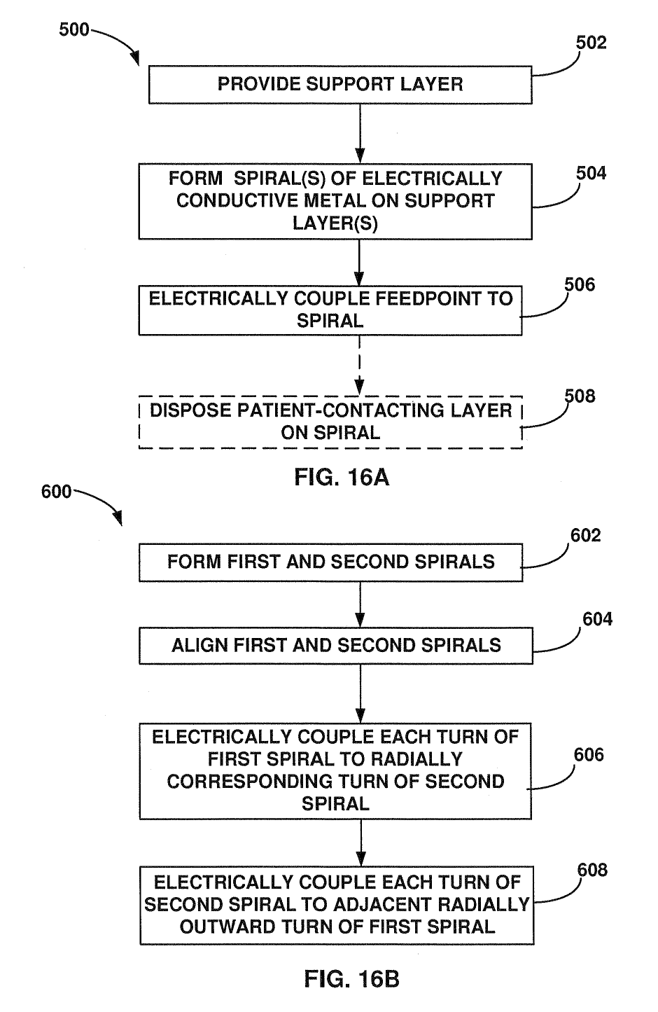

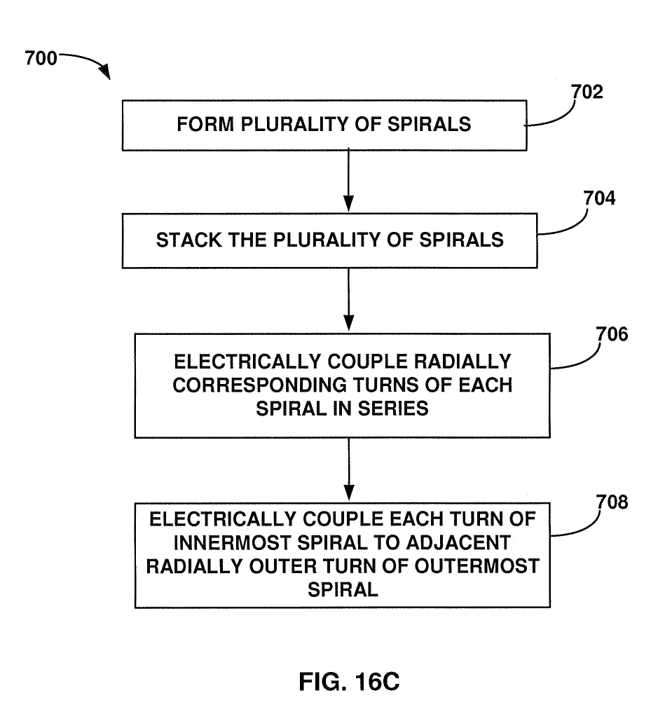

FIG. 15 is a flow chart schematically representing steps in a method 400 for making a handpiece for an electrosurgical system, according to another embodiment of the invention. The handpiece may have various components, elements, and features as described hereinabove with respect to various embodiments of the instant invention. Step 402 of method 400 may involve providing a shell and an electrode for the handpiece. The electrode may comprise a spiral inductor. The electrode may be formed according to one or more methods described with reference to FIGS. 16A-C (infra).

Step 404 may involve affixing a cooling unit to the shell. The shell may provide a housing for the electrode and the cooling unit. In an embodiment, the cooling unit may comprise a thermoelectric cooler (TEC). Step 406 may involve disposing a support layer adjacent to the cooling unit, such that the support layer may be in thermal communication with the cold side of the cooling unit. The support layer may be disposed in contact with, or adjacent to, the cooling unit. The support layer may be both electrically insulating and thermally conductive.

Step 408 may involve disposing the electrode on the support layer. In an embodiment, the electrode may be formed integrally with the support layer, such that the electrode may be disposed within the shell during step 406 (see, e.g., FIGS. 16A-C). In an embodiment, the electrode may comprise one or more spiral layers, wherein each spiral layer may comprise a spiral trace of electrically conductive metal disposed, e.g., “printed,” on a support layer to provide a substantially planar multi-layer or single layer spiral inductor (see, e.g., FIGS. 9-12B).

The handpiece may be configured for being affixed or adhered to the external skin surface of a patient. In an embodiment, the handpiece may be affixed or adhered to the skin via suction applied to the handpiece. Step 410 may involve forming at least one suction port. The at least one suction port may be configured for coupling the handpiece to a vacuum source or unit. The at least one suction port may be disposed at the periphery, edge(s), or corner(s) of the electrode.

### Methods for Making Spiral Inductors

FIG. 16A is a flow chart schematically representing steps in a method 500 for making a spiral inductor, according to another embodiment of the invention. Step 502 may involve providing a support layer. The support layer may comprise a layer or sheet of an electrically insulating or non-conductive material. In an embodiment, the support layer may be electrically insulating and thermally conductive.

Step 504 may involve forming at least one spiral of electrically conductive metal on at least one support layer. For example, in embodiments where the spiral inductor includes a plurality of spirals, each spiral of electrically conductive metal may be formed on a separate support layer. A lower portion of each spiral may be in contact with the support layer. Each spiral may be formed as a trace of the electrically conductive metal, or each spiral may be deposited on the support layer as a filament of the electrically conductive metal. In an embodiment, a metal trace forming each spiral may be formed by a printing, or printing-like, process. As a non-limiting example, one or more printing processes similar to those used for the production of flexible electrical circuits may be used in step 504. The spiral(s) formed in step 504 and described elsewhere herein according to the present invention, may be referred to as comprising a metal “trace”, regardless of the techniques or processes for forming such spiral(s). Each spiral may have an inner terminus (see, for example, FIGS. 9 and 10A-B).

Step 506 may involve electrically coupling the inner terminus of the spiral to a feedpoint, wherein the feedpoint may be configured for coupling the inner terminus to a power supply. In an embodiment where the spiral inductor comprises a plurality of spiral layers, the spirals may be electrically coupled in a specific manner (see, for example, FIGS. 12A-B, and 16B-C), in which case only the outermost spiral may be coupled to the feedpoint.

An external surface or outer portion of the spiral inductor may include a bare metal patient-contacting surface of the spiral of electrically conductive metal, which may contact the patient's body (e.g., skin) during a procedure. In some embodiments, optional step 508 may involve disposing a patient-contacting layer on the external metal surface of the spiral inductor, such that the patient-contacting layer defines a patient-contacting surface (see, e.g., FIG. 11). The patient-contacting layer may comprise an electrically conductive material having an electrical resistivity value less than 0.1 Ohm·m, and in some embodiments 0.01 Ohm·m or less.

FIG. 16B is a flow chart schematically representing steps in a method 600 for making a spiral inductor for an electrosurgical electrode, according to another embodiment of the invention. Step 602 may involve forming a first spiral and a second spiral. Each spiral may comprise a trace of electrically conductive metal disposed on an electrically insulating support layer.

Step 604 may involve aligning the first and second spirals. The first spiral may be the outermost spiral which contacts the patient, while the second spiral may be the innermost spiral of the spiral inductor. In an embodiment, the first and second spirals may have the same spiral configuration, e.g., the same number of turns with the same pitch (see, e.g., FIG. 9). During step 604, the first and second spirals may be arranged or stacked on top of each other. For example, the first and second spirals may be arranged such that the first or radially innermost turn of the second spiral is vertically aligned with the first or radially innermost turn of the first spiral.

Step 606 may involve electrically coupling each turn of the first spiral to the radially corresponding turn of the second spiral, for example, the n^{th} turn of the first spiral may be coupled to the n^{th} turn of the second spiral.

Step 608 may involve electrically coupling each turn of the second spiral to the adjacent radially outward turn of the first spiral, for example, the n^{th} turn of the second spiral may be coupled to the (n+1)^{th} turn of the second spiral. Thus, the first and second spirals may be coupled by a plurality of “vertical” connections (step 606), as well as by a plurality of “radial” connections (step 608) (see, e.g., FIG. 12A). Note that step 608 could be described in an alternative manner to provide an equivalent structure, namely: electrically coupling each turn of the first spiral to the adjacent radially inward turn of the second spiral. Method 600 may be used in combination with one or more steps of method 500 (FIG. 16A). Multi-layer spiral inductors are also disclosed in commonly assigned, co-pending U.S. patent application Ser. No. 11/966,895, entitled “High Conductivity Inductively Equalized Electrodes and Methods,” (Atty. Docket No. ALTU-3000), filed Dec. 28, 2007, the disclosure of which is incorporated by reference herein in its entirety.

FIG. 16C is a flow chart schematically representing steps in a method 700 for electrically coupling a plurality of spirals to provide a multi-layer spiral inductor, according to another embodiment of the invention. Step 702 may involve forming a plurality of spirals of electrically conductive metal. As an example, each of the spirals may be formed generally as described with reference to FIG. 16A or 16B. Each of the spirals may have elements and features as described hereinabove, e.g., with reference to one or more of FIGS. 9 and 12B.

Step 704 may involve stacking the plurality of spirals. The spirals may have identical spiral configurations, essentially as described hereinabove. The spirals may be stacked vertically, and the plurality of spirals may be aligned with each other.

Steps 706 and 708 may involve electrically coupling the plurality of spirals. The spirals may be interconnected such that each turn of the plurality of spirals is coupled to at least one other spiral. The turns of each spiral may be interconnected, for example, by connections such as vias, or the like. The interconnection of metal traces is well known in the printed circuit board art, as an example. In an embodiment, the spirals may be interconnected in a specific manner, for example, as shown in FIGS. 12A-B, to provide multi-layer spiral inductors of the instant invention. Thus, step 706 may involve electrically coupling, in series, each radially corresponding turn of each spiral. For example, each turn of a first spiral of the plurality of stacked spirals may be coupled to a radially corresponding turn of each successive one of the spirals, e.g., the n^{th} turn of each spiral may be electrically coupled in series.

Step 708 may involve electrically coupling each turn of an innermost spiral of the plurality of spirals to an adjacent, radially outward turn of the first or outermost spiral. Naturally, in a situation where the number of turns of the outermost spiral is equal to or less than the number of turns of the innermost spiral, the outermost spiral will lack a turn located radially outward from the radially outermost turn of the innermost spiral. Hence the proviso in this situation that a radially outermost turn of the innermost spiral is not coupled to an adjacent, radially outward turn of the outermost spiral. But no such proviso applies in a situation where the number of turns of the outermost spiral is greater than the number of turns of the innermost spiral.

### Monitoring Electrosurgical Procedures

FIG. 17A schematically represents a handpiece, as seen from the side, according to one aspect of the invention. Handpiece 50 may include elements substantially as described hereinabove, including void 59 within shell 51, flange 58, and an electrode 60 disposed within void 59. Handpiece 50 may further include at least one temperature sensor 54. Temperature sensor(s) 54 may be disposed within void 59.

Handpiece 50 may be in fluid communication, via suction port 72, with a vacuum unit 70. Handpiece 50 may further include at least one pressure sensor 80 disposed within void 59. Under the invention, pressure sensor(s) 80 may be disposed at alternative locations in the vacuum path between handpiece 50 and vacuum unit 70. Handpiece 50 may be configured for applying suction to the patient's skin, SK, via vacuum unit 70; and void 59 may be configured for receiving a region of the patient's skin. In an embodiment, subcutaneous fat, SF, may also be received by void 59.

In FIG. 17A, handpiece 50 is shown as being disposed against the patient's skin, such that flange 58 contacts the external surface, ES, of the skin. In FIG. 17A, suction port(s) 72 may be disconnected from vacuum unit 70 (see, e.g., FIG. 17B), and/or vacuum unit 70 may be idle (off). Accordingly, in FIG. 17A void 59 does not contain tissue of the patient.

In FIG. 17B, suction port(s) 72 may be connected to vacuum unit 70 and vacuum unit 70 may be activated (on). Flange 58 may be adapted for sealing engagement with the external surface of the skin. For example, flange 58 may be configured for sealing void 59 against the skin (with or without the application of a sealing material to the skin and/or flange 58). Accordingly, in FIG. 17B tissue of the patient may be drawn into void 59, by suction applied from vacuum unit 70, such that electrode 60 may make contact with the external surface of the patient's skin. In an embodiment, a region of subcutaneous fat may be drawn into void 59 in addition to the skin. Electrodes 60 may be disposed substantially centrally, and recessed within, void 59 such that the patient's tissue/skin contacts electrode 60 when the target tissue is drawn into void 59. Pressure sensors 80 may be configured for sensing pressure values within void 59, so that patient contact with electrode 60 may be monitored, via sensed pressure values, during a procedure. As an example, pressure sensor(s) 80 may indicate an abrupt pressure increase if patient contact with handpiece 50/electrode 60 is broken.

Electrode 60 may be disposed on an electrically insulating and thermally conductive support layer 52; and support layer 52 may be disposed against a cooling unit 56, such that electrode 60 is in thermal communication with cooling unit 56 (see, e.g., FIG. 6C). Temperature sensors 54 may be disposed adjacent to electrode 60, and temperature sensors 54 may be configured for sensing skin temperature during a procedure.

FIG. 18 is a flow chart schematically representing steps in a method 800 for monitoring patient-electrode contact during an electrosurgical procedure, according to another embodiment of the invention. Step 802 may involve contacting a patient's body with at least one electrosurgical handpiece. The handpiece may be adapted for treating the patient's skin, subcutaneous tissue, cellulite, or the like, substantially as described with reference to FIG. 14A, supra. The handpiece may include various elements and features as described hereinabove (e.g., with reference to FIGS. 5-7B), including a shell defining a void, an electrode disposed in the void, and at least one suction port in communication with the void. The handpiece may further include at least one pressure sensor configured for sensing pressure values within the void (see, e.g., FIGS. 17A-B). The pressure sensor(s) may be disposed adjacent to the electrode (see, e.g., FIG. 7B).

Step 804 may involve applying a vacuum to the handpiece, via the suction port, wherein an area of skin of the patient's body may be drawn into the void, such that the skin may contact the electrode. Step 806 may involve sensing pressure values within the void. Such pressure values may be sensed via the pressure sensor(s). Step 808 may involve monitoring, via the sensed pressure values, contact between the electrode and the skin. As a non-limiting example, an increase in sensed pressure value may indicate lack of sealing engagement between the handpiece and the patient, which may result in lack of contact between the electrode and the patient's skin.

Step 810 may involve providing a warning signal, in response to an increase in sensed pressure values above a threshold pressure level, which may indicate lack of contact between the electrode and the patient's skin. In the event of such a signal, the procedure may be interrupted pending corrective action being taken by an operator of the handpiece/electrosurgical system. While the handpiece is in contact with the patient's body, e.g., during steps 804-808, an AC voltage may be applied to the electrode sufficient to remove or modify at least a portion of the targeted tissue.

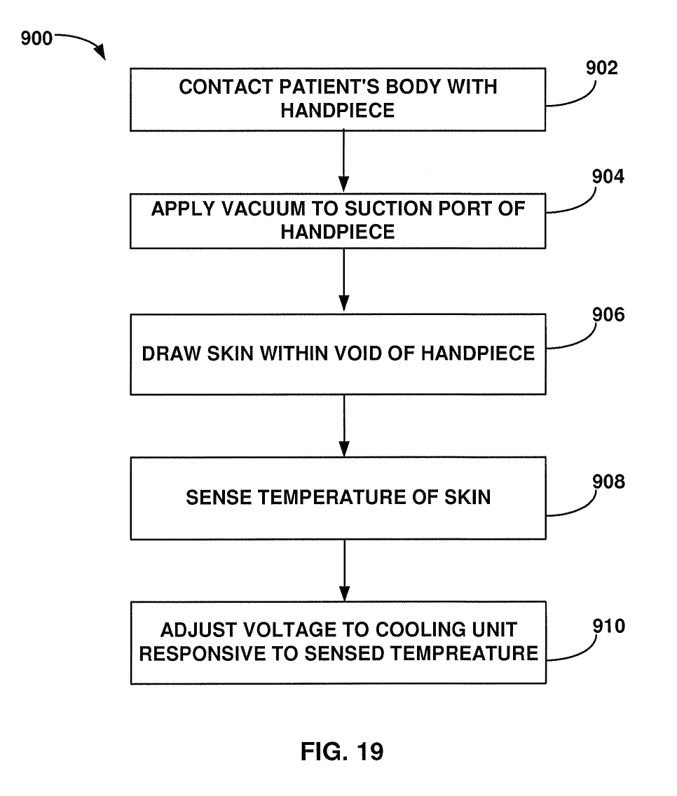

According to another aspect of the invention, the electrosurgical handpiece that is brought in contact with the patient in step 802 may further include at least one temperature sensor (see, e.g., FIGS. 4A and 7B). While the handpiece is in contact with the patient during a procedure, skin temperature may be sensed via one or more temperature sensors, and skin temperature may be controlled by adjusting the voltage to the cooling unit (see, e.g., method 900, FIG. 19).

FIG. 19 is a flow chart schematically representing steps in a method 900 for controlling skin temperature during an electrosurgical procedure, according to another embodiment of the invention. Step 902 may involve contacting a patient's body with at least one electrosurgical handpiece. The handpiece may be adapted for treating the patient's skin, subcutaneous tissue, cellulite, or the like, substantially as described with reference to FIG. 14A, supra. The handpiece may include various elements and features as described hereinabove (e.g., with reference to FIGS. 5-7B and 18), including a shell defining a void of the handpiece and an electrode recessed within the shell. The handpiece may further include at least one temperature sensor configured for sensing temperature values of the skin, and a cooling unit configured for cooling the skin. The temperature sensor(s) may be disposed within the void and adjacent to the electrode (see, e.g., FIG. 7B).

Step 904 may involve applying a vacuum to the handpiece, via the suction port whereby suction may be applied to an area of skin of the patient's body. Step 906 may involve drawing the area of skin or tissue to be treated within the void of the handpiece. The area of skin of the patient's body may be drawn into the void by the applied suction such that the skin may contact both the electrode and the temperature sensor(s). While the electrode is in contact with the area of skin corresponding to a target tissue, an AC voltage may be applied to the electrode to provide electrical energy to the patient's body sufficient to remove or modify at least a portion of the target tissue. The target tissue may comprise, for example, an area of skin that is in contact with the electrode, or subcutaneous tissue beneath the area of skin. In an embodiment, the procedure may use a system having two handpieces (see, e.g., FIG. 1A) or two electrode-bearing pads (see, e.g., FIG. 8A), and the AC voltage may be applied to the electrodes substantially as described with reference to FIGS. 14A-B.

Step 908 may involve sensing, via the temperature sensor(s), temperature values of the skin. Thus, skin temperature may be monitored during application of electrical energy to the target tissue via the electrode. Step 910 may involve adjusting a voltage applied to the cooling unit in response to the temperature values sensed in step 908. In an embodiment, the cooling unit may comprise a thermoelectric cooler, whereby an increase in voltage may increase cooling of the patient's skin (via the Peltier effect).

It is to be understood that the foregoing relates to exemplary embodiments of the invention, and that methods and apparatus of the invention may find many applications other than those specifically described herein. None of the examples presented herein are to be construed as limiting the present invention in any way; modifications may be made without departing from the spirit and scope of the invention as set forth in the following claims.

1-59. (canceled)

60. A system for treating a patient, comprising:
an electrosurgical generator;
a first handpiece coupled to said electrosurgical generator; and
a second handpiece coupled to said electrosurgical generator, wherein:
said system is configured for providing a first AC voltage to said first handpiece and for providing a second AC voltage to said second handpiece; and
said first handpiece and said second handpiece are manipulable separately from each other.

61. The system of claim 60, wherein:
each of said first handpiece and said second handpiece includes a substantially planar electrode; and
each of said first handpiece and said second handpiece is configured for contacting an area of an external surface of the skin of the patient, wherein the area is at least about 10 cm².

62. The system of claim 60, further comprising:
at least one vacuum unit configured for providing suction to each of said first handpiece and said second handpiece, wherein each of said first handpiece and said second handpiece comprises a shell defining a void within each of said first handpiece and said second handpiece; and
each of said first handpiece and said second handpiece includes at least one pressure sensor configured for sensing pressure at least one location along a vacuum path between said vacuum unit and said shell.

63. The system of claim 60, wherein:
said first handpiece includes a first electrode electrically coupled to said electrosurgical generator; and
said second handpiece includes a second electrode electrically coupled to said electrosurgical generator, wherein:
said electrosurgical generator is configured for providing said first AC voltage to said first electrode;
said electrosurgical generator is further configured for concurrently providing said second AC voltage to said second electrode; and
said first and second AC voltages are of equal magnitude and opposite polarity.

64. The system of claim 63, wherein:
each of said first electrode and said second electrode comprises a spiral inductor; and
each said spiral inductor comprises at least one spiral of electrically conductive metal disposed on an electrically insulating support layer.

65. The system of claim 60, wherein said electrosurgical generator is configured for providing a phase difference of about 180° between said first and second AC voltages.

66. The system of claim 60, wherein:
each of said first handpiece and said second handpiece is configured for separately contacting an external surface of the skin of the patient;
said first handpiece and said second handpiece are movable independently of each other; and
said system is configured for varying a separation distance between a first skin location of said first handpiece and a second skin location of said second handpiece.

67. The system of claim 63, further comprising:
at least one temperature sensor configured for sensing temperature values of the skin or a target tissue of the patient; and
wherein said system is configured for independently controlling power delivery to each of said first electrode and said second electrode in response to said sensed temperature values.

68. The system of claim 60, wherein:
each of said first and second handpieces includes a cooling unit and a temperature sensor;
each of said first and second handpieces is configured for sensing temperature values of the skin or a target tissue of the patient; and
said system is configured for controlling a voltage applied to said cooling unit in response to said sensed temperature values.

69. The system of claim 63, wherein:
said first handpiece comprises a first electrode-bearing pad and said second handpiece comprises a second electrode-bearing pad;
said first electrode is disposed on said first electrode-bearing pad and said second electrode is disposed on said second electrode-bearing pad; and
each of said first and second electrode bearing pads is flexible.

70-71. (canceled)

72. A handpiece for treating a patient, comprising:
a shell including a central planar portion;
a planar electrode recessed within said shell, wherein said electrode is disposed substantially parallel to said central planar portion;
said shell including at least one suction port; and
a collar portion extending distally from said central planar portion, wherein said shell is at least substantially frusto-pyramidal or frusto-conical, said shell defining a void within said handpiece, wherein:
said handpiece is configured for applying suction, via said suction port, to tissue of the patient; and
said handpiece is further configured for receiving the tissue of the patient within said void, such that an external surface of the skin contacts said electrode.

73. The handpiece of claim 72, further comprising:
a cooling unit in thermal communication with said electrode; and
a support layer disposed between said cooling unit and said electrode, wherein said support layer comprises a thermally conductive and electrically insulating material.

74. The handpiece of claim 72, further comprising:
at least one pressure sensor disposed within said void; and
at least one temperature sensor disposed adjacent to said electrode.

75. The handpiece of claim 72, wherein:
said electrode comprises a spiral inductor;
said spiral inductor comprises a spiral trace of electrically conductive metal; and
said spiral trace of electrically conductive metal occupies from about 60% to 99% of the external surface area of said spiral inductor.

76. The handpiece of claim 75, wherein:
said spiral inductor comprises a plurality of stacked spirals; and
said plurality of spirals are electrically interconnected by a plurality of vias.

77. A method for treating a patient, comprising:
a) providing a first AC voltage to a first electrode of an electrosurgical system;
b) providing a second AC voltage to a second electrode of said electrosurgical system, wherein step b) is performed concurrently with step a), and said first and second AC voltages are of substantially equal magnitude and opposite polarity, whereby a potential difference is provided between said first and second electrodes; and
c) via said first and second electrodes, applying electrical energy to a target tissue of the patient, wherein said electrical energy is sufficient to remove or modify at least a portion of the target tissue.

78. The method of claim 77, wherein said first and second electrodes each comprise a spiral inductor.

79. The method of claim 77, wherein:
said first electrode is disposed on a first handpiece;
said second electrode is disposed on a second handpiece;
each of said first handpiece and said second handpiece is configured for contacting the skin of the patient; and
said first and second handpieces are separately movable with respect to each other.

80. The method of claim 79, further comprising:
d) disposing said first and second handpieces at separate locations on the skin of the patient, wherein:
said first electrode is disposed at a first skin location;
said second electrode is disposed at a second skin location;
the target tissue is disposed between the first skin location and the second skin location; and
said first and second AC voltages have a phase difference of about 180°.

81. The method of claim 80, wherein:
each of said first and second handpieces includes at least one suction port; and
step d) comprises affixing said first handpiece to the skin at the first skin location of the patient, and affixing said second handpiece to the skin at the second skin location of the patient.

82. The method of claim 80, wherein:
the target tissue comprises subcutaneous fat; and
at least one of the first and second skin locations is on a part of the patient's body selected from the group consisting of the abdomen, the back, the buttocks, the hips, the thighs, the upper arms, and the neck.

83-90. (canceled)
